(19) United States
(12) Patent Application Publication
(10) Pub. No.: US 2023/0331856 A1
(43) Pub. Date: Oct. 19, 2023

(54) MATERIALS AND METHODS TO REDUCE PROTEIN AGGREGATION

(71) Applicant: AMGEN INC., Thousand Oaks, CA (US)

(72) Inventors: Arnold J. MCAULEY, Thousand Oaks, CA (US); Michael SCHNEIDER, Thousand Oaks, CA (US)

(21) Appl. No.: 18/044,158

(22) PCT Filed: Sep. 10, 2021
(86) PCT No.: PCT/US2021/049872

§ 371 Date: Mar. 6, 2023

Related U.S. Application Data
(60) Provisional application No. 63/077,088, filed on Sep. 11, 2020.

(51) Int. Cl. A61P 35/04; C07K 16/28

(52) U.S. Cl. CPC C07K 16/2863; A61P 35/04; C07K 16/2809; C07K 2317/31

(57) ABSTRACT

Provided herein are methods of treating a neoplastic disease in a subject. In exemplary embodiments, the method comprises administering to the subject an aqueous solution comprising an anti-EGFRvIII agent using an administration system, wherein one or more of the components of the administration system, or parts thereof, which contact the aqueous solution substantially lack a polyvinyl chloride (PVC) and/or air. In exemplary aspects, the administration system is an infusion system comprising an infusion line, wherein at least part of the infusion line substantially lacks PVC. In exemplary aspects, the administration system comprises a container for containing the aqueous solution wherein less than about 5% of the volume of the container is air, when the container comprises the aqueous solution. Related kits are further provided herein.

### CROSS-REFERENCE TO RELATED APPLICATIONS

The benefit under 35 U.S.C. §119(e) of U.S. Provisional Application No. 63/077,088, filed on Sep. 11, 2020, is hereby claimed, and the disclosure thereof is hereby incorporated herein by reference.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 122,414 bytes ASCII (Text) file named “A-2651-WO-PCT_Sequence_Listing.txt”; created on Sep. 10, 2021.

### Field of the Invention

The present invention relates to administration of anti-EGFRvIII agents for the treatment of a neoplastic disease, e.g., cancer.

### Background of the Invention

Glioblastomas (GBM) belong to the group of highly malignant brain tumors representing one of the most lethal human cancers. The age-adjusted incidence of glioblastoma ranges from 0.59 to 3.69 per 100,000 persons worldwide. Despite aggressive surgical, radiologic and chemotherapeutic intervention, tumors progress within months or even weeks leading to an overall survival of 12 to 15 months with almost no change in prognosis since the FDA’s approval of temozolomide (TMZ) in 2005 (Omuro & DeAngelis, JAMA, 2013;310:1842-1850).

Upon recurrence after primary surgery, management of glioblastoma depends on age, performance status, histology, initial therapy response, time from original diagnosis, and whether the occurrence is local or diffuse. In patients with diffuse or multiple tumor recurrences, palliative care is a common choice. In patients with localized disease, combination of surgery, nitrosourea-based therapies, and radiation (standard re-irradiation or highly conformal radiation) is used, with poor results. A response to chemotherapy is unlikely after 2 consecutive agents have failed to produce a response (Stewart et al., Lancet 2002;359(9311): 1011-1018). Moreover, no survival benefit has since been demonstrated for any new agent in a randomized clinical study (Mehta et al, Crit Rev Oncol Hematol. 2017;111: 60-65).

Epidermal growth factor receptor (EGFR) expression and enhanced EGF pathway signaling activity accompanied by amplification of the gene encoding EGFR have been documented in glioblastoma, almost exclusively in isocitrate dehydrogenase (IDH) wildtype glioblastoma (Louis et al., Acta Neuropathol. 2016;131:803-820). About 50% of glioblastomas are positive for EGFR amplification, half of which express the accompanying EGFR mutation, encoding a truncated and constitutively active receptor termed EGFRvIII (Epidermal Growth Factor Receptor Variant III). Like native EGFR, mutant EGFRvIII is a membrane-bound receptor; however the deletion results in a protein lacking 267 amino acid residues encompassing the extracellular ligand binding domain and characterized by a novel glycine residue occurring at the splice junction (Wong et al., Proc Natl Acad Sci USA. 1992;89:2965-2969). While lacking an extracellular ligand binding domain, EGFRvIII has shown ligand-independent constitutive tyrosine kinase activity that stimulates downstream signaling pathways, which promote malignant growth (Mellinghoff et al., N Engl J Med. 2005;353:2012-2024). According to one meta-analysis (Chen et al., Acta Neurol Scand. 2015;132:310-322), there is currently insufficient evidence that either EGFR amplification or the EGFRvIII mutation has prognostic value in patients with glioblastoma. EGFRvIII is nevertheless considered a bona-fide tumor-specific antigen found exclusively on tumor cells thereby making it an attractive antitumor treatment strategy.

### SUMMARY

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).

E1. A method of treating a neoplastic disease in a subject, comprising administering to the subject an aqueous solution comprising an anti-EGFRvIII agent using an administration system, wherein one or more of the components of the administration system, or one or more portions of the component(s), which contact the aqueous solution substantially lack(s) a polyvinyl chloride (PVC) or PVC plasticizer and/or substantially lack air.

E2. The method of E1, wherein the administration system is an infusion system.

E3. The method of E2, wherein the infusion system comprises an infusion line, wherein at least a portion of the infusion line substantially lacks a PVC or PVC plasticizer.

E4. The method of E3, wherein the portion of the infusion line that contacts the aqueous solution substantially lacks PVC, optionally, wherein the face of the interior of the infusion line that contacts the aqueous solution substantially lacks a PVC or PVC plasticizer.

E5. The method of E3 or E4, wherein the infusion line comprises a polyolefin elastomer (POE) or polyurethane (PUR) or ethylene vinyl acetate (EVA).

E6. The method of E5, wherein the infusion line is composed entirely of the POE, PUR, or EVA, or the face of the interior of the infusion line is coated with POE, PUR, or EVA.

E7. The method of any one of the preceding embodiments, wherein the administration system comprises a container for containing the aqueous solution.

E8. The method of E7, wherein at least part of the container substantially lacks a PVC or PVC plasticizer, optionally, wherein the part of the container that contacts the aqueous solution substantially lacks a PVC or PVC plasticizer.

E9. The method of E7 or E8, wherein, when the container comprises the aqueous solution, the container substantially lacks air.

E10. The method of E8, wherein, when the container comprises the aqueous solution, less than about 5% of the volume of the container is air.

E11. The method of E9, wherein, when the container comprises the aqueous solution, less than about 3% of the volume of the container is air.

E12. The method of any one of the preceding embodiments, wherein the aqueous solution is administered to the subject by intravenous (IV) infusion, optionally, continuous intravenous infusion (clVi).

E13. The method of E12, wherein the anti-EGFRvIII agent is administered to the subject as a clVi at a constant flow rate.

E14. The method of any one of E2-E13, wherein the infusion system further comprises an infusion line filter, an intravenous (IV) bag, an infusion pump, or a combination thereof.

E15. The method of E14, wherein the infusion line filter is a polyethylsulfone (PES) filter.

E16. The method of E14 or E15, wherein, when the IV bag comprises the aqueous solution, the IV bag substantially lacks air.

E17. The method of E16, wherein less than about 5% of the volume of the IV bag is air, when the container comprises the aqueous solution.

E18. The method of E17, wherein less than about 3% of the volume of the IV bag is air, when the container comprises the aqueous solution.

E19. The method of any one or E14 to E18, wherein at least part of the IV bag substantially lacks a PVC or PVC plasticizer, optionally, wherein the portion of the IV bag that contacts the aqueous solution substantially lacks a PVC or PVC plasticizer.

E20. The method of E19, wherein at least a portion of the IV bag comprises an ethylene vinyl acetate (EVA) or a polyolefin elastomer (POE).

E21. The method E20, wherein the IV bag is composed entirely of the EVA or POE or the face of the interior of the IV bag is lined with EVA or POE.

E22. The method of any one of E14 to E21, wherein the infusion pump is a CADD pump.

E23. The method of any one of E14 to E22, wherein the infusion pump is programmable, lockable, non-elastomeric, and/or has an alarm.

E24. The method of any one of the preceding embodiments, wherein the aqueous solution is infused at an infusion speed of about 1.5 ml/hour to about 150 mL/hour, optionally, about 1.5 mL per hour to about 5 mL/hour.

E25. The method of any one of the preceding embodiments, wherein the average cumulative subvisible particle counts/mL of the post-administration solution is less than 15, as determined by a liquid particle counter.

E26. The method of claim E25, wherein the average cumulative subvisible particle counts/mL of the post-administration solution is less than 10, optionally, less than 1, as determined by a liquid particle counter.

E27. The method of any one of the preceding embodiments, wherein, upon visual inspection, the post-administration solution has less than 5 visible particles greater than or equal to 125 µm.

E28. The method of any one of the preceding embodiments, wherein the % high molecular weight (HMW) species of the post-administration solution is less than 5%, as determined by size exclusion-high performance liquid chromatography (SE-HPLC).

E29. The method of any one of the preceding embodiments, wherein the % high molecular weight (HMW) species of the post-administration solution is less than 2% and/or the % main peak of the anti-EGFRvIII agent is greater than 95%, as determined by SE-HPLC.

E30. The method of any one of the preceding embodiments, wherein the % recovery of the anti-EGFRvIII agent in the post-administration solution is greater than 95% as determined by ultraviolet2-visible (UV-VIS) spectroscopy or reversed phase-high performance liquid chromatography (RP-HPLC).

E31. The method of any one of the preceding embodiments, wherein the neoplastic disease is a cancer or a tumor.

E32. The method of E31, wherein the cancer or tumor is an EGFRvlll-positive cancer or tumor.

E32. The method of E31 or E32, wherein said cancer is a squamous cell tumor, such as non-small cell lung cancer (NSCLC).

E33. The method of E31 or E32, wherein said cancer is glioblastoma or malignant glioma.

E34. The method of any one of the embodiments, wherein the aqueous solution comprises saline, optionally, about 0.5% to about 1.5% (v/v) saline.

E35. The method of any one of the preceding embodiments, wherein the solution comprises about 2% (v/v) to about 6% (v/v) intravenous saline solution (IVSS), optionally, about 4% (v/v) IVSS.

E36. The method of any one of the preceding embodiments, wherein the anti-EGFRvIII agent is present in the aqueous solution at a concentration of less than 75 ng/mL, optionally, less than 50 ng/mL.

E37. The method of E36, wherein the anti-EGFRvIII agent is present in the aqueous solution at a concentration of about 75 ng/mL to about 500 ng/mL, optionally, about 200 ng/mL to about 400 ng/mL.

E38. The method of any one of the preceding embodiments, wherein the final volume of the aqueous solution is about 100 mL to about 300 mL solution.

E39. The method of any one of the preceding embodiments, wherein said anti-EGFRvIII agent is a bispecific antigen-binding polypeptide comprising: a first binding domain that binds to human EGFRvIII and macaque EGFRvIII, and a second binding domain that binds to human CD3.

E40. The method of E39, wherein said human EGFRvIII comprises the amino acid sequence of SEQ ID NO:1, and said macaque EGFRvIII comprises an amino acid sequence of SEQ ID NO:2.

E41. The method of E39 or E40, wherein said human CD3 comprises residues 1-27 of SEQ ID NO:123.

E42. The method of any one of E39-E41, wherein said human CD3 comprises the amino acid sequence of SEQ ID NO:123.

E43. The method of any one of E39-E42, wherein said EGFRvIII-binding domain comprises: (a) a heavy chain variable region (VH) that comprises: (i) a VH complementarity determining region one (CDR-H1) comprising the amino acid sequence of SEQ ID NO:3; (ii) a CDR-H2 comprising the amino acid sequence of SEQ ID NO:4; and (iii) a CDR-H3 comprising the amino acid sequence of SEQ ID NO:5; and (b) a light chain variable region (VL) that comprises: (i) a VL complementarity determining region one (CDR-L1) comprising the amino acid sequence of SEQ ID NO:6; (ii) a CDR-L2 comprising the amino acid sequence of SEQ ID NO:7; and (iii) a CDR-L3 comprising the amino acid sequence of SEQ ID NO:8.

E44. The method of any one of E39-E43, wherein said EGFRvIII-binding domain comprises: a VH that comprises the amino acid sequence of SEQ ID NO:9, and a VL that comprises the amino acid sequence of SEQ ID NO:10.

E45. The method of E43 or E44, wherein said VH and VL are joined by a linker to form a single chain Fv (scFv).

E46. The method of E45, wherein said linker is a peptide linker comprising a sequence selected from any one of SEQ ID Nos. 114-122.

E47. The method of E45 or E46, wherein said linker comprises (Gly4Ser)x, where x is an integer of 1, 2, 3, or 4.

E48. The method of any one of E39-E47, wherein said EGFRvIII-binding domain comprises the amino acid sequence of SEQ ID NO:11.

E49. The method of any one of E39-E48, wherein said CD3-binding domain comprises:
a. a VH that comprises: a CDR-H1 comprising the amino acid sequence of SEQ ID NO:18, a CDR-H2 comprising the amino acid sequence of SEQ ID NO:19, and a CDR-H3 comprising the amino acid sequence of SEQ ID NO:20; and a VL that comprises: a CDR-L1 comprising the amino acid sequence of SEQ ID NO:15, a CDR-L2 comprising the amino acid sequence of SEQ ID NO:16, and a CDR-L3 comprising the amino acid sequence of SEQ ID NO:17;
b. a VH that comprises: a CDR-H1 comprising the amino acid sequence of SEQ ID NO:27, a CDR-H2 comprising the amino acid sequence of SEQ ID NO:28, and a CDR-H3 comprising the amino acid sequence of SEQ ID NO:29; and a VL that comprises: a CDR-L1 comprising the amino acid sequence of SEQ ID NO:24, a CDR-L2 comprising the amino acid sequence of SEQ ID NO:25, and a CDR-L3 comprising the amino acid sequence of SEQ ID NO:26;
c. a VH that comprises: a CDR-H1 comprising the amino acid sequence of SEQ ID NO:36, a CDR-H2 comprising the amino acid sequence of SEQ ID NO:37, and a CDR-H3 comprising the amino acid sequence of SEQ ID NO:38; and a VL that comprises: a CDR-L1 comprising the amino acid sequence of SEQ ID NO:33, a CDR-L2 comprising the amino acid sequence of SEQ ID NO:34, and a CDR-L3 comprising the amino acid sequence of SEQ ID NO:35;
d. a VH that comprises: a CDR-H1 comprising the amino acid sequence of SEQ ID NO:45, a CDR-H2 comprising the amino acid sequence of SEQ ID NO:46, and a CDR-H3 comprising the amino acid sequence of SEQ ID NO:47; and a VL that comprises: a CDR-L1 comprising the amino acid sequence of SEQ ID NO:42, a CDR-L2 comprising the amino acid sequence of SEQ ID NO:43, and a CDR-L3 comprising the amino acid sequence of SEQ ID NO:44;
e. a VH that comprises: a CDR-H1 comprising the amino acid sequence of SEQ ID NO:54, a CDR-H2 comprising the amino acid sequence of SEQ ID NO:55, and a CDR-H3 comprising the amino acid sequence of SEQ ID NO:56; and a VL that comprises: a CDR-L1 comprising the amino acid sequence of SEQ ID NO:51, a CDR-L2 comprising the amino acid sequence of SEQ ID NO:52, and a CDR-L3 comprising the amino acid sequence of SEQ ID NO:53;
f. a VH that comprises: a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; and a VL that comprises: a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62;
g. a VH that comprises: a CDR-H1 comprising the amino acid sequence of SEQ ID NO:72, a CDR-H2 comprising the amino acid sequence of SEQ ID NO:73, and a CDR-H3 comprising the amino acid sequence of SEQ ID NO:74; and a VL that comprises: a CDR-L1 comprising the amino acid sequence of SEQ ID NO:69, a CDR-L2 comprising the amino acid sequence of SEQ ID NO:70, and a CDR-L3 comprising the amino acid sequence of SEQ ID NO:71;
h. a VH that comprises: a CDR-H1 comprising the amino acid sequence of SEQ ID NO:81, a CDR-H2 comprising the amino acid sequence of SEQ ID NO:82, and a CDR-H3 comprising the amino acid sequence of SEQ ID NO:83; and a VL that comprises: a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80;
i. a VH that comprises: a CDR-H1 comprising the amino acid sequence of SEQ ID NO:90, a CDR-H2 comprising the amino acid sequence of SEQ ID NO:91, and a CDR-H3 comprising the amino acid sequence of SEQ ID NO:92; and a VL that comprises: a CDR-L1 comprising the amino acid sequence of SEQ ID NO:87, a CDR-L2 comprising the amino acid sequence of SEQ ID NO:88, and a CDR-L3 comprising the amino acid sequence of SEQ ID NO:89;
j. a VH that comprises: a CDR-H1 comprising the amino acid sequence of SEQ ID NO:99, a CDR-H2 comprising the amino acid sequence of SEQ ID NO:100, and a CDR-H3 comprising the amino acid sequence of SEQ ID NO:101; and a VL that comprises: a CDR-L1 comprising the amino acid sequence of SEQ ID NO:96, a CDR-L2 comprising the amino acid sequence of SEQ ID NO:97, and a CDR-L3 comprising the amino acid sequence of SEQ ID NO:98; OR
k. a VH that comprises: a CDR-H1 comprising the amino acid sequence of SEQ ID NO:108, a CDR-H2 comprising the amino acid sequence of SEQ ID NO:109, and a CDR-H3 comprising the amino acid sequence of SEQ ID NO:110; and a VL that comprises: a CDR-L1 comprising the amino acid sequence of SEQ ID NO:105, a CDR-L2 comprising the amino acid sequence of SEQ ID NO:106, and a CDR-L3 comprising the amino acid sequence of SEQ ID NO:107.

E50. The method of any one of E39-E49, wherein said CD3-binding domain comprises:
a. a VH that comprises the amino acid sequence of SEQ ID NO:21, and a VL that comprises the amino acid sequence of SEQ ID NO:22;
b. a VH that comprises the amino acid sequence of SEQ ID NO:30, and a VL that comprises the amino acid sequence of SEQ ID NO:31;
c. a VH that comprises the amino acid sequence of SEQ ID NO:39, and a VL that comprises the amino acid sequence of SEQ ID NO:40;
d. a VH that comprises the amino acid sequence of SEQ ID NO:48, and a VL that comprises the amino acid sequence of SEQ ID NO:49;
e. a VH that comprises the amino acid sequence of SEQ ID NO:57, and a VL that comprises the amino acid sequence of SEQ ID NO:58;
f. a VH that comprises the amino acid sequence of SEQ ID NO:66, and a VL that comprises the amino acid sequence of SEQ ID NO:67;
g. a VH that comprises the amino acid sequence of SEQ ID NO:75, and a VL that comprises the amino acid sequence of SEQ ID NO:76;
h. a VH that comprises the amino acid sequence of SEQ ID NO:84, and a VL that comprises the amino acid sequence of SEQ ID NO:85;
i. a VH that comprises the amino acid sequence of SEQ ID NO:93, and a VL that comprises the amino acid sequence of SEQ ID NO:94;
j. a VH that comprises the amino acid sequence of SEQ ID NO:102, and a VL that comprises the amino acid sequence of SEQ ID NO:103; or
k. a VH that comprises the amino acid sequence of SEQ ID NO:111, and a VL that comprises the amino acid sequence of SEQ ID NO:112.

E51. The method of any one of E49 or E50, wherein said, the VH and VL of the CD3-binding domain are joined by a linker to form a single chain Fv (scFv).

E52. The method of E51, wherein said linker is a peptide linker comprising a sequence selected from any one of SEQ ID Nos. 114-122.

E53. The method of E51 or E52, wherein said linker comprises (Gly4Ser)x, where x is an integer of 1, 2, 3, or 4.

E54. The method of any one of E39-E53, wherein said CD3-binding domain comprises the amino acid sequence of any one of SEQ ID NOs:23, 32, 41, 50, 59, 68, 77, 86, 95, 104, and 113.

E55. The method of any one of E39-E54, wherein:
said EGFRvIII-binding domain comprises: (a) a heavy chain variable region (VH) that comprises: (i) a VH complementarity determining region one (CDR-H1) comprising the amino acid sequence of SEQ ID NO:3; (ii) a CDR-H2 comprising the amino acid sequence of SEQ ID NO:4; and (iii) a CDR-H3 comprising the amino acid sequence of SEQ ID NO:5; and (b) a light chain variable region (VL) that comprises: (i) a VL complementarity determining region one (CDR-L1) comprising the amino acid sequence of SEQ ID NO:6; (ii) a CDR-L2 comprising the amino acid sequence of SEQ ID NO:7; and (iii) a CDR-L3 comprising the amino acid sequence of SEQ ID NO:8; and
said CD3-binding domain comprises: (a) a heavy chain variable region (VH) that comprises: (i) a VH complementarity determining region one (CDR-H1) comprising the amino acid sequence of SEQ ID NO:99; (ii) a CDR-H2 comprising the amino acid sequence of SEQ ID NO:100; and (iii) a CDR-H3 comprising the amino acid sequence of SEQ ID NO:101; and (b) a light chain variable region (VL) that comprises: (i) a VL complementarity determining region one (CDR-L1) comprising the amino acid sequence of SEQ ID NO:96; (ii) a CDR-L2 comprising the amino acid sequence of SEQ ID NO:97; and (iii) a CDR-L3 comprising the amino acid sequence of SEQ ID NO:98.

E56. The method of any one of E39-E55, wherein said EGFRvIII-binding domain comprises: a VH that comprises the amino acid sequence of SEQ ID NO:9, and a VL that comprises the amino acid sequence of SEQ ID NO:10; and wherein said CD3-binding domain comprises: a VH that comprises the amino acid sequence of SEQ ID NO:102, and a VL that comprises the amino acid sequence of SEQ ID NO:103.

E57. The method of any one of E39-E56, wherein said EGFRvIII-binding domain comprises the amino acid sequence of SEQ ID NO:11, and said CD3-binding domain comprises the amino acid sequence of SEQ ID NO:104.

E58. The method of any one of E39-E57, wherein said EGFRvIII-binding domain and said CD3-binding domain are joined by a linker.

E59. The method of E58, wherein said linker is a peptide linker comprising a sequence selected from any one of SEQ ID Nos. 114-122.

E60. The method of E58 or E59, wherein said linker comprises (Gly4Ser)x, where x is an integer of 1, 2, 3, or 4.

E61. The method of any one of the preceding embodiments, wherein said anti-EGFRvIII agent comprises the amino acid sequence of SEQ ID NO: 12.

E62. The method of any one of the preceding embodiments, wherein said anti-EGFRvIII agent comprises the amino acid sequence of SEQ ID NO: 13.

E63. The method of any one E1-E62, wherein said anti-EGFRvIII agent is administered for at least 14 days at the initial dose of from about 15 µg/day to about 12000 µg/day.

E64. The method of any one E1-E63, wherein said anti-EGFRvIII agent is administered for at least 14 days at the initial dose of from about 1500 µg/day to about 6000 µg/day.

E65. The method of any one E1-E64, wherein said anti-EGFRvIII agent is administered for at least 14 days at the initial dose of from about 3000 µg/day to about 6000 µg/day.

E66. The method of any one E1-E65, wherein said anti-EGFRvIII agent is administered for at least 28 days at the initial dose of from about 15 µg/day to about 12000 µg/day.

E67. The method of any one E1-E66, wherein said anti-EGFRvIII agent is administered for at least 28 days at the initial dose of from about 1500 µg/day to about 6000 µg/day.

E68. The method of any one E1-E67, wherein said anti-EGFRvIII agent is administered for at least 28 days at the initial dose of from about 3000 µg/day to about 6000 µg/day.

E69. The method of any one of E1-E68, wherein said anti-EGFRvIII agent is administered at a 14-day on / 14-day off cycle, or a 28-day on / 14-day off cycle.

E70. A kit comprising an administration system and an anti-EGFRvIII agent, wherein one or more of the components of the administration system, or at least a portion of the component(s), which contact the aqueous solution, substantially lack a PVC or PVC plasticizer and/or air.

E71. The kit of E70, wherein the administration system is an infusion system comprising an infusion line, wherein at least a portion of the infusion line substantially lacks PVC or PVC plasticizer.

E72. The kit of E70 or E71, wherein the administration system comprises a container for containing the aqueous solution wherein less than about 5% of the volume of the container is air, when the container comprises the aqueous solution.

E73. An article of manufacture comprising an aqueous solution comprising an anti-EGFRvIII agent in a container wherein less than about 5% of the volume of the container is air, when the container comprises the aqueous solution.

E74. A kit comprising an IV bag comprising an aqueous solution comprising about 3% (v/v) to about 6% (v/v) IVSS and saline and a container comprising an anti-EGFRvIII agent, wherein at least a portion of the IV bag which contacts the aqueous solution, substantially lacks a PVC or PVC plasticizer.

### BRIEF DESCRIPTION OF THE DRAWINGS

FIG. 1 is an image of the particulation of an anti-EGFRvIII agent (AMG 596) in an IV bag after agitation.

FIGS. 2A and 2B are images of an aqueous solution comprising an anti-EGFRvIII agent (AMG 596) in an IV bag (250-mL MediBag) without air (FIG. 2A) or with air (FIG. 2B).

Each of FIGS. 2C and 2D is a graph of the particle counts/mL of an aqueous solution comprising AMG 596 in the indicated IV bag for the indicated particle size.

FIG. 2E is a graph of the % protein recovery of AMG 596 in the indicated IV bag with or without air. AMG 596 recovery was close to 100% for all IV bags when no air was present in the IV bag.

FIG. 3A is a flow chart of the experimental steps followed with an aqueous solution comprising an anti-EGFRvIII agent (AMG 596), as described herein. FIG. 3B is a table listing the details of the IV bag preparation.

Each of FIGS. 4A and 4B is a graph of the cumulative particle counts/mL of an aqueous solution comprising an anti-EGFRvIII agent (AMG 596) passed through an infusion system with or without an inline filter. Particle count for 10 µm particles is shown in FIG. 4A and particle count for 25 µm particles is shown in FIG. 4B.

Each of FIGS. 5A and 5B is a graph of the cumulative particle counts/mL of an aqueous solution comprising an anti-EGFRvIII agent (AMG 596) passed through an infusion system at an infusion speed of 5 mL/hour or 125 mL/hour. Particle count for 25 µm particles is shown in FIG. 5A and particle count for 10 µm particles is shown in FIG. 5B.

### DETAILED DESCRIPTION

### 1. Overview

AMG 596 is a bispecific T-cell engaging protein that binds to EGFRvIII and CD3 to activate T cells and bring them in proximity of glioblastoma cells to kill these cells. AMG 596 is a very potent molecule with a short half-life. Because the half-life is very short, the protein is administered through continuous intravenous infusion (clVi) to maintain a sufficient level of protein in a patient to be efficacious. The clVi administration setup is usually made up of at least an IV bag, an infusion pump, and an infusion line. The AMG 596 drug product (DP) is in some embodiments admixed in an IV bag with saline and an intravenous solution stabilizer (IVSS) to make the infusion solution. This solution is then infused into a patient by being pumped with an infusion pump through an infusion line into the patient. Based on an evaluation of infusion systems, and components and materials thereof, the present disclosure provides efficient and improved methods of administering AMG 596 for the treatment of a patient with a neoplastic disease.

### 2. Definitions

An antigen-binding polypeptide according to the present invention is preferably a polypeptide which immunospecifically binds to its target or antigen. It typically comprises the heavy chain variable region (VH) and/or the light chain variable region (VL) of an antibody, or comprises domains derived therefrom. A polypeptide according to the invention comprises the minimum structural requirements of an antibody which allow for immunospecific target binding. This minimum requirement may e.g. be defined by the presence of at least three light chain CDRs (i.e. CDR1, CDR2 and CDR3 of the VL region) and/or three heavy chain CDRs (i.e. CDR1, CDR2 and CDR3 of the VH region), preferably of all six CDRs. It is within the knowledge of a skilled person where (in which order) those CDRs are located in the binding domain.

A T-cell engaging antigen-binding polypeptide according to the present inventio, such as a BiTE^{®} molecule, is preferably bispecific which is understood herein to typically comprise one domain binding to at least one target antigen and another domain binding to CD3. Hence, it does not occur naturally, and it is markedly different in its function from naturally occurring products. A polypeptide in accordance with the invention is hence an artificial “hybrid” polypeptide comprising at least two distinct binding domains with different specificities and is, thus, bispecific. It is emphasized that bispecific T-cell engaging polypeptides disclosed herein may comprise more than two domains, e.g., they may comprise two identical or different target antigen binding domains and another domain binding to CD3. The target antigen binding domains may be identical, i.e. bind the same epitope, or they may bind different epitopes of the same of different target antigens. A T-cell engaging polypeptide may be characterized by the presence of three or six CDRs in either one or both binding domains.

Exemplary bispecific anti-EGFRvIII agents disclosed herein (such as BiTE^{®} molecules) are antigen-binding polypeptides comprising two or more antigen-binding domains, one domain binding to EGFRvIII, and another binding to CD3. In exemplary embodiments, each binding domain comprises a scFv (VL and VH domains of a single arm of an antibody connected via a linker). Although germline VL and VH domains are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. Science 242:423- 426 (1988) and Huston et al., 1988, Proc. Natl. Acad. Sci. USA 85:5879-5883.

A “variable domain” refers to the variable region of the antibody light chain (VL) or the variable region of the antibody heavy chain (VH), either alone or in combination. As known in the art, the variable regions of the heavy and light chains each consist of four framework regions (FR) connected by three complementarity determining regions (CDRs), and contribute to the formation of the antigen-binding site of antibodies.

The “Complementarity Determining Regions” (CDRs) of exemplary EGFRvlll-binding domains and CD3-binding domains are provided in the Sequence Table. The CDRs can be defined according to Kabat, Chothia, the accumulation of both Kabat and Chothia, AbM, contact, North, and/or conformational definitions or any method of CDR determination well known in the art. See, e.g., Kabat et al., 1991, Sequences of Proteins of Immunological Interest, 5th ed. (hypervariable regions); Chothia et al., 1989, Nature 342:877-883 (structural loop structures). AbM definition of CDRs is a compromise between Kabat and Chothia and uses Oxford Molecular’s AbM antibody modeling software (Accelrys^{®}). The identity of the amino acid residues in a particular antibody that make up a CDR can be determined using methods well known in the art.

“About” or “approximately,” when used in connection with a measurable numerical variable, refers to the indicated value of the variable and to all values of the variable that are within the experimental error of the indicated value (e.g. within the 95% confidence interval for the mean) or ±10% of the indicated value, whichever is greater. Numeric ranges are inclusive of the numbers defining the range.

### 3. Anti-EGFRvIII Agents

The epidermal growth factor receptor (EGFR) is a pivotal regulator of normal cellular growth in tissues of epithelial origin. Dysregulated EGFR signaling (resulting from mechanisms such as cell-surface overexpression, autocrine activation and EGFR gene mutation) contributes to the formation of many epithelial malignancies in humans. Several EGFR mutations have been described. The most common extracellular mutation is EGFRvIII (also known as de2-7EGFR and ΔEGFR). EGFRvIII is a tumor-specific mutation that results from in-frame deletion of 801 base pairs spanning exons 2-7 of the coding sequence. This deletion removes 267 amino acids from the extracellular domain, creating a junction site between exons 1 and 8 and a new glycine residue. EGFRvIII has a molecular mass of approximately 145 kDa. The amino acid sequences of human and cynomolgus EGFRvIII are shown as SEQ ID Nos. 1 and 2, respectively.

An exemplary anti-EGFRvIII agent is a bispecific molecule that binds EGFRvIII and CD3, such as a BiTE^{®} (bispecific T cell engager) molecule. BiTE^{®} molecules are recombinant proteins made from two flexibly linked binding domains, each domain derived from antibodies. One binding domain of BiTE^{®} molecule is specific for a tumor-associated surface antigen (such as EGFRvIII); the second binding domain is specific for CD3, a subunit of the T cell receptor complex on T cells. By their design, BiTE^{®} molecules are uniquely suited to transiently connect T cells with target cells and, at the same time, potently activate the inherent cytolytic potential of T cells against target cells. See e.g., WO 99/54440, WO 2005/040220, and WO 2008/119567.

Accordingly, in some embodiments, the anti-EGFRvIII agent described comprises two binding domains: the first domain binds EGFRvIII (preferably human EGFRvIII), and the second domain binds CD3 (preferably human CD3). Exemplary CD3 sequences are provided as SEQ ID Nos. 123-126. Preferably, the second domain binds to residues 1-27 of SEQ ID NO:123. Alternatively, the second domain may bind to residues 1-27 of any one of SEQ ID NOs:124-126.

In certain embodiments, the EGFRvIII-binding domain comprises: (a) a heavy chain variable region (VH) that comprises: (i) a VH complementarity determining region one (CDR-H1) comprising the amino acid sequence of SEQ ID NO:3; (ii) a CDR-H2 comprising the amino acid sequence of SEQ ID NO:4; and (iii) a CDR-H3 comprising the amino acid sequence of SEQ ID NO:5; and (b) a light chain variable region (VL) that comprises: (i) a VL complementarity determining region one (CDR-L1) comprising the amino acid sequence of SEQ ID NO:6; (ii) a CDR-L2 comprising the amino acid sequence of SEQ ID NO:7; and (iii) a CDR-L3 comprising the amino acid sequence of SEQ ID NO:8.

In certain embodiments, the EGFRvIII-binding domain comprises: a VH that comprises the amino acid sequence of SEQ ID NO:9, and a VL that comprises the amino acid sequence of SEQ ID NO:10. In some embodiments, the VH and VL are joined by a linker to form a single chain Fv (scFv). In some embodiments, the linker is a peptide linker comprising a sequence selected from any one of SEQ ID Nos. 114-122. In some embodiments, the linker is a GS liker, such as Gly-Gly-Gly-Gly-Ser (G4S, SEQ ID NO: 115), or polymers thereof, i.e. (Gly4Ser)x, where x is an integer of 1 or greater (e.g. 2 or 3) (e.g., SEQ ID Nos. 121, 122).

In certain embodiments, the EGFRvIII-binding domain comprises the amino acid sequence of SEQ ID NO:11.

In certain embodiments, the CD3-binding domain comprises:
(a) a VH that comprises: a CDR-H1 comprising the amino acid sequence of SEQ ID NO:18, a CDR-H2 comprising the amino acid sequence of SEQ ID NO:19, and a CDR-H3 comprising the amino acid sequence of SEQ ID NO:20; and a VL that comprises: a CDR-L1 comprising the amino acid sequence of SEQ ID NO:15, a CDR-L2 comprising the amino acid sequence of SEQ ID NO:16, and a CDR-L3 comprising the amino acid sequence of SEQ ID NO:17;
(b) a VH that comprises: a CDR-H1 comprising the amino acid sequence of SEQ ID NO:27, a CDR-H2 comprising the amino acid sequence of SEQ ID NO:28, and a CDR-H3 comprising the amino acid sequence of SEQ ID NO:29; and a VL that comprises: a CDR-L1 comprising the amino acid sequence of SEQ ID NO:24, a CDR-L2 comprising the amino acid sequence of SEQ ID NO:25, and a CDR-L3 comprising the amino acid sequence of SEQ ID NO:26;
(c) a VH that comprises: a CDR-H1 comprising the amino acid sequence of SEQ ID NO:36, a CDR-H2 comprising the amino acid sequence of SEQ ID NO:37, and a CDR-H3 comprising the amino acid sequence of SEQ ID NO:38; and a VL that comprises: a CDR-L1 comprising the amino acid sequence of SEQ ID NO:33, a CDR-L2 comprising the amino acid sequence of SEQ ID NO:34, and a CDR-L3 comprising the amino acid sequence of SEQ ID NO:35;
(d) a VH that comprises: a CDR-H1 comprising the amino acid sequence of SEQ ID NO:45, a CDR-H2 comprising the amino acid sequence of SEQ ID NO:46, and a CDR-H3 comprising the amino acid sequence of SEQ ID NO:47; and a VL that comprises: a CDR-L1 comprising the amino acid sequence of SEQ ID NO:42, a CDR-L2 comprising the amino acid sequence of SEQ ID NO:43, and a CDR-L3 comprising the amino acid sequence of SEQ ID NO:44;
(e) a VH that comprises: a CDR-H1 comprising the amino acid sequence of SEQ ID NO:54, a CDR-H2 comprising the amino acid sequence of SEQ ID NO:55, and a CDR-H3 comprising the amino acid sequence of SEQ ID NO:56; and a VL that comprises: a CDR-L1 comprising the amino acid sequence of SEQ ID NO:51, a CDR-L2 comprising the amino acid sequence of SEQ ID NO:52, and a CDR-L3 comprising the amino acid sequence of SEQ ID NO:53;
(f) a VH that comprises: a CDR-H1 comprising the amino acid sequence of SEQ ID NO:63, a CDR-H2 comprising the amino acid sequence of SEQ ID NO:64, and a CDR-H3 comprising the amino acid sequence of SEQ ID NO:65; and a VL that comprises: a CDR-L1 comprising the amino acid sequence of SEQ ID NO:60, a CDR-L2 comprising the amino acid sequence of SEQ ID NO:61, and a CDR-L3 comprising the amino acid sequence of SEQ ID NO:62;
(g) a VH that comprises: a CDR-H1 comprising the amino acid sequence of SEQ ID NO:72, a CDR-H2 comprising the amino acid sequence of SEQ ID NO:73, and a CDR-H3 comprising the amino acid sequence of SEQ ID NO:74; and a VL that comprises: a CDR-L1 comprising the amino acid sequence of SEQ ID NO:69, a CDR-L2 comprising the amino acid sequence of SEQ ID NO:70, and a CDR-L3 comprising the amino acid sequence of SEQ ID NO:71;
(h) a VH that comprises: a CDR-H1 comprising the amino acid sequence of SEQ ID NO:81, a CDR-H2 comprising the amino acid sequence of SEQ ID NO:82, and a CDR-H3 comprising the amino acid sequence of SEQ ID NO:83; and a VL that comprises: a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80;
(i) a VH that comprises: a CDR-H1 comprising the amino acid sequence of SEQ ID NO:90, a CDR-H2 comprising the amino acid sequence of SEQ ID NO:91, and a CDR-H3 comprising the amino acid sequence of SEQ ID NO:92; and a VL that comprises: a CDR-L1 comprising the amino acid sequence of SEQ ID NO:87, a CDR-L2 comprising the amino acid sequence of SEQ ID NO:88, and a CDR-L3 comprising the amino acid sequence of SEQ ID NO:89;
(j) a VH that comprises: a CDR-H1 comprising the amino acid sequence of SEQ ID NO:99, a CDR-H2 comprising the amino acid sequence of SEQ ID NO:100, and a CDR-H3 comprising the amino acid sequence of SEQ ID NO:101; and a VL that comprises: a CDR-L1 comprising the amino acid sequence of SEQ ID NO:96, a CDR-L2 comprising the amino acid sequence of SEQ ID NO:97, and a CDR-L3 comprising the amino acid sequence of SEQ ID NO:98; OR
(k) a VH that comprises: a CDR-H1 comprising the amino acid sequence of SEQ ID NO:108, a CDR-H2 comprising the amino acid sequence of SEQ ID NO:109, and a CDR-H3 comprising the amino acid sequence of SEQ ID NO:110; and a VL that comprises: a CDR-L1 comprising the amino acid sequence of SEQ ID NO:105, a CDR-L2 comprising the amino acid sequence of SEQ ID NO:106, and a CDR-L3 comprising the amino acid sequence of SEQ ID NO:107.

In certain embodiments, the CD3-binding domain comprises:
(a) a VH that comprises the amino acid sequence of SEQ ID NO:21, and a VL that comprises the amino acid sequence of SEQ ID NO:22;
(b) a VH that comprises the amino acid sequence of SEQ ID NO:30, and a VL that comprises the amino acid sequence of SEQ ID NO:31;
(c) a VH that comprises the amino acid sequence of SEQ ID NO:39, and a VL that comprises the amino acid sequence of SEQ ID NO:40;
(d) a VH that comprises the amino acid sequence of SEQ ID NO:48, and a VL that comprises the amino acid sequence of SEQ ID NO:49;
(e) a VH that comprises the amino acid sequence of SEQ ID NO:57, and a VL that comprises the amino acid sequence of SEQ ID NO:58;
(f) a VH that comprises the amino acid sequence of SEQ ID NO:66, and a VL that comprises the amino acid sequence of SEQ ID NO:67;
(g) a VH that comprises the amino acid sequence of SEQ ID NO:75, and a VL that comprises the amino acid sequence of SEQ ID NO:76;
(h) a VH that comprises the amino acid sequence of SEQ ID NO:84, and a VL that comprises the amino acid sequence of SEQ ID NO:85;
(i) a VH that comprises the amino acid sequence of SEQ ID NO:93, and a VL that comprises the amino acid sequence of SEQ ID NO:94;
(j) a VH that comprises the amino acid sequence of SEQ ID NO:102, and a VL that comprises the amino acid sequence of SEQ ID NO:103; or
(k) a VH that comprises the amino acid sequence of SEQ ID NO:111, and a VL that comprises the amino acid sequence of SEQ ID NO:112.

In some embodiments, the VH and VL of the CD3-binding domain are joined by a linker to form a single chain Fv (scFv). In some embodiments, the linker is a peptide linker comprising a sequence selected from any one of SEQ ID Nos. 114-122. In some embodiments, the linker is a GS liker, such as Gly-Gly-Gly-Gly-Ser (G4S, SEQ ID NO: 115), or polymers thereof, i.e. (Gly4Ser)x, where x is an integer of 1 or greater (e.g. 2 or 3) (e.g., SEQ ID Nos. 121, 122).

In certain embodiments, the CD3-binding domain comprises the amino acid sequence of any one of SEQ ID NOs:23, 32, 41, 50, 59, 68, 77, 86, 95, 104, and 113.

In certain embodiments, the EGFRvIII-binding domain and the CD3-binding domain are joined by a linker. In some embodiments, the linker is a peptide linker comprising a sequence selected from any one of SEQ ID Nos. 114-122. In some embodiments, the linker is a GS liker, such as Gly-Gly-Gly-Gly-Ser (G4S, SEQ ID NO: 115), or polymers thereof, i.e. (Gly4Ser)x, where x is an integer of 1 or greater (e.g. 2 or 3) (e.g., SEQ ID Nos. 121, 122).

In certain embodiments, the anti-EGFRvIII agent described herein comprises two domains. The first domain binds EGFRvIII (preferably human EGFRvIII) and comprises: (a) a heavy chain variable region (VH) that comprises: (i) a VH complementarity determining region one (CDR-H1) comprising the amino acid sequence of SEQ ID NO:3; (ii) a CDR-H2 comprising the amino acid sequence of SEQ ID NO:4; and (iii) a CDR-H3 comprising the amino acid sequence of SEQ ID NO:5; and (b) a light chain variable region (VL) that comprises: (i) a VL complementarity determining region one (CDR-L1) comprising the amino acid sequence of SEQ ID NO:6; (ii) a CDR-L2 comprising the amino acid sequence of SEQ ID NO:7; and (iii) a CDR-L3 comprising the amino acid sequence of SEQ ID NO:8. The second domain binds CD3 (preferably human CD3) and comprises: (a) a heavy chain variable region (VH) that comprises: (i) a VH complementarity determining region one (CDR-H1) comprising the amino acid sequence of SEQ ID NO:99; (ii) a CDR-H2 comprising the amino acid sequence of SEQ ID NO:100; and (iii) a CDR-H3 comprising the amino acid sequence of SEQ ID NO:101; and (b) a light chain variable region (VL) that comprises: (i) a VL complementarity determining region one (CDR-L1) comprising the amino acid sequence of SEQ ID NO:96; (ii) a CDR-L2 comprising the amino acid sequence of SEQ ID NO:97; and (iii) a CDR-L3 comprising the amino acid sequence of SEQ ID NO:98.

In certain embodiments, the anti-EGFRvIII agent described herein comprises two domains: (a) the first domain binds EGFRvIII (preferably human EGFRvIII) and comprises: a VH that comprises the amino acid sequence of SEQ ID NO:9, and a VL that comprises the amino acid sequence of SEQ ID NO:10; and (b) the second domain binds CD3 (preferably human CD3) and comprises: a VH that comprises the amino acid sequence of SEQ ID NO:102, and a VL that comprises the amino acid sequence of SEQ ID NO:103.

In certain embodiments, the anti-EGFRvIII agent described herein comprises two domains: (a) the first domain binds EGFRvIII (preferably human EGFRvIII) and comprises the amino acid sequence of SEQ ID NO:11; and (b) the second domain binds CD3 (preferably human CD3) and comprises the amino acid sequence of SEQ ID NO:104.

In certain embodiments, the anti-EGFRvIII agent described herein comprises the amino acid sequence of SEQ ID NO: 12. In certain embodiments, the anti-EGFRvIII agent described herein comprises the amino acid sequence of SEQ ID NO: 13.

Preferably, the anti-EGFRvIII agent is administered parenterally (e.g., intravenously) and then can cross the blood brain barrier (BBB). Without wishing to be bound by a particular theory, it is believed that the binding of CD3 contributes the penetration of BBB by the exemplary anti-EGFRvIII agents described herein. Activated T lymphocytes are known to have the ability to penetrate the BBB under normal physiological conditions. By binding to CD3 on the surface of T cell, it is believed that the exemplary anti-EGFRvIII agents can activate peripheral circulating T cells, thereby passing through the BBB via these T cells.

Methods to produce the anti-EGFRvIII agents are well known to the skilled artisan. For example, it is known that for “single chain Fv” (scFv) the two domains of the Fv fragment, VL and VH, are coded for by separate genes, but they can be joined, using recombinant methods, by an artificial linker - as described hereinbefore - that enables them to be made as a single protein chain in which the VL and VH regions pair to form a monovalent molecule; see e.g., Huston et al. (1988) Proc. Natl. Acad. Sci USA 85:5879-5883). These antibody fragments and derivatives are obtained using conventional techniques known to those with skill in the art, and the fragments are evaluated for function in the same manner as are full-length antibodies or IgGs. A single-chain variable fragment (scFv) is hence a fusion protein of the variable region of the heavy chain (VH) and of the light chain (VL) of immunoglobulins, usually connected with a short linker peptide. The linker is usually rich in glycine for flexibility, as well as serine or also threonine for solubility (as described herein above). This protein retains the specificity of the original immunoglobulin, despite removal of the constant regions and introduction of the linker.

Techniques described for producing single chain antibody-derived polypeptides (see, inter alia, U.S. Pat. 4,946,778, Kontermann and Dübel (2010), loc. cit. and Little (2009), loc. cit.) can be adapted to produce an antigen-binding polypeptide specifically recognizing (an) elected target(s).

Bivalent (also called divalent) or bispecific single-chain variable fragments (bi-scFvs or di-scFvs) having the format (scFv)₂ can be engineered by linking two scFv molecules (e.g. with linkers as described hereinbefore). The linking can be done by producing a single polypeptide chain with two VH regions and two VL regions, yielding tandem scFvs (see e.g. Kufer P. et al., (2004) Trends in Biotechnology 22(5):238-244).

### 4. Methods of Treatment

The present disclosure provides methods of administering an anti-EGFRvIII agent for treatment of a neoplastic disease in a subject and methods of treating a neoplastic disease in a subject. In exemplary embodiments, the method comprises administering to the subject a composition (e.g., an aqueous solution) comprising an anti-EGFRvIII agent using an administration system, wherein one or more of the components of the administration system, or at least a portion of the component(s), which contact the composition (e.g., aqueous solution), substantially lack(s) a polyvinyl chloride (PVC) or PVC plasticizer and/or substantially lack(s) air. In exemplary embodiments, the method comprises administering to the subject a composition (e.g., an aqueous solution) comprising an anti-EGFRvIII agent using an infusion system comprising an infusion line, wherein the infusion line is not a PVC infusion line or which substantially lacks a PVC or PVC plasticizer. Optionally, the infusion line is composed entirely of a material other than PVC. Alternatively, the interior of the infusion line is coated with a material other than PVC. In exemplary embodiments, the method comprises administering to the subject a composition (e.g., an aqueous solution) comprising an anti-EGFRvIII agent using an administration system (e.g., an infusion system) comprising an intravenous (IV) bag wherein less than 5% of the total volume of the IV bag is air, when the IV bag comprises the composition (e.g., aqueous solution) comprising the anti-EGFRvIII agent.

As used herein the term “administration system” is synonymous with “drug administration system” and refers to a device, or a set of components used together, for administering a drug or therapeutic agent (e.g., an anti-EGFRvIII agent) to a subject (e.g., a human subject). An administration system, in exemplary aspects, comprises one or more of a syringe, a needle, a pen, inhaler, eye dropper, a catheter, a container (e.g., an intravenous (IV) container, an IV bag, a vial, a cartridge) and the like. In various aspects, the administration system is an infusion system for administering a drug or therapeutic agent to a subject by infusion (e.g., intravenous infusion, subcutaneous infusion). In various aspects, the infusion system is purposed for intravenous administration of the drug or therapeutic agent. Optionally, the infusion system comprises an IV administration set used to administer fluids from a container to a subject’s vascular system through a needle or catheter inserted into a vein. The IV administration set, in various aspects, comprises one or more of: a needle, catheter, tubing, flow regulator, drip chamber, infusion line, infusion line filter, IV set stopcock, fluid delivery tubing, connectors between parts of the set, side tube with a cap to serve as an injection site, infusion pump, infusion port, and/or hollow spike to penetrate and connect the tubing to an IV bag or other infusion fluid container. In various instances, the IV administration set comprises an infusion line and/or an IV bag.

In various aspects, one or more of the components of the administration system (e.g., infusion system), or at least a portion of the component(s), which contact the aqueous solution, substantially lack(s) PVC or a PVC plasticizer. PVC is a synthetic plastic polymer produced by the polymerization of vinyl chloride monomers and is generally a rigid, non-pliable material. In various instances, PVC is mixed with a plasticizer to make it more flexible and/or durable. In various aspects, the PVC is a plasticized PVC. In exemplary instances, one or more of the components of the administration system (e.g., infusion system), or at least a portion of the component(s), which contact the aqueous solution, substantially lack(s) PVC (e.g., a plasticized PVC) or a PVC plasticizer (e.g., an ester plasticizer, phthalate plasticizer). In various aspects, the one or more portions comprise(s) a polymer other than PVC, e.g., other than a plasticized PVC. In various aspects, one or more of the components of the administration system (e.g., infusion system), or at least a portion of the component(s), which contact the aqueous solution, comprises less than about 5% PVC, e.g., less than about 5% plasticized PVC. Optionally, one or more of the components of the administration system (e.g., infusion system), or at least a portion of the component(s), which contact the aqueous solution, comprises less than about 4%, less than about 3%, less than about 2%, or less than about 1% PVC, e.g., plasticized PVC. In exemplary aspects, one or more of the components of the administration system (e.g., infusion system), or at least a portion of the component(s), which contact the aqueous solution, comprises less than about 0.9%, less than about 0.8%, less than about 0.7%, less than about 0.6%, less than about 0.5%, less than about 0.4%, less than about 0.3%, less than about 0.2%, or less than about 0.1% PVC, e.g., plasticized PVC.

In various aspects, one or more of the components of the administration system (e.g., infusion system), or at least a portion of the component(s), which contact the aqueous solution, comprise(s) polyolefin elastomer (POE) or polyurethane (PUR) or ethylene vinyl acetate (EVA). Optionally, one or more of the components of the administration system (e.g., infusion system), or at least a portion of the component(s), which contact the aqueous solution, is composed entirely of the POE, PUR or EVA. Alternatively, one or more of the components of the administration system (e.g., infusion system), comprise(s) plasticized PVC yet the portion(s) which contact(s) the aqueous solution are coated or lined with a material which substantially lacks PVC or a PVC plasticizer, optionally, the one or more portions which contact(s) the aqueous solution are coated or lined with POE, PUR or EVA. In various aspects, at least the interior of the infusion line is coated with a material other than PVC (e.g., plasticized PVC), optionally, at least the interior of the infusion line is coated or lined with POE, PUR or EVA. In various aspects, the POE is a copolymer of either ethylene-butene or ethylene-octene. In some aspects, the POE is and ethylene/polypropylene elastomer (EPM). Optionally, the POE is poly-4-methyl-1-pentene. In various aspects, the POE is an ENGAGE™ POE, an INFUSE™ POE, AFFINITY™GA POE, or NORDEL™ EPDM POE (Dow Chemical Co., Midland, MI). Additional POEs are known in the art. See, e.g., U.S. Pat. Application Publication No. 20180163024, McKeen, Chapter 9 “Polyolefins”, Film Properties of Plastics and Elastomers (4^{th} ed.), Plastics Design Library, William Andrew Applied Science Publishers, pgs. 229-272 (2017). In exemplary instances, the PUR is any polymer composed of organic units joined by carbamate (urethane) links. Optionally, the PUR is made from a di- or tri-isocyanate and a polyol. In various aspects, the isocyanate is an aromatic diisocyanate, toluene diisocyante, or a methylene diphenyl diisocyanate. In some instances, the isocyanate is isocyanates are 1,6-hexamethylene diisocyanate (HDI), 1-isocyanato-3-isocyanatomethyl-3,5,5-trimethyl-cyclohexane (isophorone diisocyanate, IPDI), and 4,4′-diisocyanato dicyclohexylmethane, (H₁₂MDI or hydrogenated MDI). In various aspects, the polyol is a polyether polyol. In various aspects, the EVA is a copolymer of ethylene and vinyl acetate. In various aspects, the weight percent of vinyl acetate is about 10% to about 40% and the remainder is ethylene. In exemplary aspects, the EVA comprises not more than 4% vinyl acetate. In alternative aspects, the EVA comprises about 4% to about 30% vinyl acetate, while in other aspects, the EVA comprises more than 60% vinyl acetate. In various aspects, the EVA comprises about 30% to about 60% vinyl acetate. In exemplary aspects, the EVA is an Elvax™ EVA copolymer resin (Dow, Midland, MI), DuPont Vamac™ EVA (DuPont, Wilmington, DE), Pre-Elec^{®} CP 1319 EVA (Omnexus, Zurich, Switzerland), Evazote^{®} EVA copolymer foam (Zotefoams Plc, Croydon, UK)), Neolene EH (Zotefoams), or an APIZERO^{®} or APIFIVE^{®} EVA compound (Trinseo, Auburn Hills, MI).

In various aspects, one or more components of the administration system (e.g., infusion system), or at least a portion of the component(s), which contact the aqueous solution, substantially lack(s) a PVC plasticizer. Plasticizers are substances added to a material to make it softer and more pliable or flexible. Plasticizers may make the material less viscous or decreases friction during the handling of the material in manufacture. Plasticizers are commonly added to PVC to increase the flexibility, pliability, and/or durability of the PVC. As used herein, the term “PVC plasticizer” refers to a plasticizer added to PVC to increase the flexibility, durability, and/or pliability of PVC. Without being bound to a particular theory, the PVC plasticizer is chemically reactive with one or more components of the aqueous solution, e.g., polysorbate 80, and causes formation of precipitates or particles in the aqueous solution. In various aspects, one or more components of the administration system (e.g., infusion system), or at least a portion of the component(s), which contact the aqueous solution, comprises less than about 5% PVC plasticizer. Optionally, one or more of the components of the administration system (e.g., infusion system), or at least a portion of the component(s), which contact the aqueous solution, comprises less than about 4%, less than about 3%, less than about 2%, or less than about 1% PVC plasticizer. In exemplary aspects, one or more of the components of the administration system (e.g., infusion system), or at least a portion of the component(s), which contact the aqueous solution, comprises less than about 0.9%, less than about 0.8%, less than about 0.7%, less than about 0.6%, less than about 0.5%, less than about 0.4%, less than about 0.3%, less than about 0.2%, or less than about 0.1% PVC plasticizer. In some instances, one or more components of the administration system (e.g., infusion system), or at least a portion of the component(s), which contact the aqueous solution, substantially lack(s) a phthalate, such as, e.g., bis(2-ethylhexyl) phthalate (DEHP), bis(2-propylheptyl) phthalate, diisononyl phthalate, di-n-butyl phthalate, butyl benzyl phthalate, diisodecyl phthalate, dioctyl phthalate, diisoctyl phthalate, diethyl phthalate, diisobutyl phthalate, di-n-hexyl phthalate, and the like. In alternative or additional aspects, one or more components of the administration system (e.g., infusion system), or at least a portion of the component(s), which contact the aqueous solution, substantially lack(s) a trimellitate, e.g., trioctyl trimellitate (TOTM), trimethyl trimellitate, tri-(n-oxtyl, n-decyl) trimellitate, tri-(hdptyl, nonyl) trimellitate, n-octyl trimellitate. In alternative or additional aspects, one or more components of the administration system (e.g., infusion system), or at least a portion of the component(s), which contact the aqueous solution, substantially lack(s) a adipate, sebacate, or maleate, e.g., bis(2-ethyldexyl)adipate, dimethyl adipate, monomethyla dipate, dioctyl adipate, dibutyl sebacate, dibutyl maleate, diisobutyl maleate.

In exemplary aspects, the method comprises administering to the subject an aqueous solution comprising an anti-EGFRvIII agent using an infusion system comprising an infusion line. In various aspects, the infusion line or portions thereof that contact the aqueous solution substantially lack(s) PVC or a PVC plasticizer, e.g., comprises less than about 5% PVC or PVC plasticizer. In various aspects, the infusion line comprises a polymer other than PVC. In exemplary instances, the infusion line comprises POE, PUR, and/or EVA. In various aspects, the infusion line is composed entirely of the polymer other than PVC, e.g., POE, PUR, EVA. Alternatively, the infusion line comprises PVC, e.g., plasticized PVC, yet the portion(s) of the infusion line which contact(s) the aqueous solution is/are coated with a material other than PVC, e.g., plasticized PVC. Optionally, the one or more portions which contact(s) the aqueous solution, e.g., the interior face of the infusion line, are coated with POE or PUR or EVA. Optionally, the administration system comprises a container for containing the aqueous solution and at least part of the container substantially lacks PVC, e.g., comprises less than about 5% PVC or PVC plasticizer.

In various embodiments, the administration system comprises a container for containing the aqueous solution and when the container comprises the aqueous solution at least part of the container substantially lacks air. In various instances, less than about 5% of the volume of the container is air, when the container comprises the aqueous solution. Optionally, less than about 4%, less than about 3%, less than about 2%, or less than about 1% of the volume of the container is air, when the container comprises the aqueous solution. In various aspects, at least part of the IV bag substantially lacks PVC or PVC plasticizer, optionally, wherein the part of the IV bag that contacts the aqueous solution substantially lacks PVC or PVC plasticizer. In some instances, at least part of the IV bag comprises an ethylene vinyl acetate (EVA) or a polyolefin elastomer (POE). For instance, the IV bag is composed entirely of the EVA or POE.

In exemplary aspects, the method comprises administering to the subject an aqueous solution comprising an anti-EGFRvIII agent using an infusion system comprising an IV bag. In some aspects, when the IV bag comprises the aqueous solution, less than about 5% of the volume of the container is air. Optionally, less than about 4%, less than about 3%, less than about 2%, or less than about 1% of the volume of the IV bag is air, when the IV bag comprises the aqueous solution. In various aspects, at least part of the IV bag substantially lacks PVC or PVC plasticizer, optionally, wherein the part of the IV bag that contacts the aqueous solution substantially lacks PVC or PVC plasticizer. In some instances, at least part of the IV bag comprises an ethylene vinyl acetate (EVA) or a polyolefin elastomer (POE). For instance, the IV bag is composed entirely of the EVA or POE.

In exemplary embodiments, the aqueous solution is administered to the subject by intravenous (IV) infusion, optionally, continuous intravenous infusion (clVi). In exemplary instances, the anti-EGFRvIII agent is administered to the subject as a clVi at a constant flow rate. In some aspects, the aqueous solution is infused at an infusion speed of about 1.5 ml/hour to about 150 mL/hour. In various instances, the aqueous solution is infused at an infusion speed of about 1.5 ml/hour to about 5 mL/hour.

The infusion system in various aspects further comprises an infusion line filter, an intravenous (IV) bag, an infusion pump, or a combination thereof. Optionally, the infusion line filter is a polyethylsulfone (PES) filter. In various instances, the infusion pump is a CADD pump. Optionally, the infusion pump is programmable, lockable, non-elastomeric, and/or has an alarm.

Without being bound to any particular theory, the methods of the present disclosure provide reduced particulation of the anti-EGFRvIII agent during infusion such that a sufficient and/or efficacious amount of the agent is provided to the subject without substantial loss of the agent. The qualities of the aqueous solution administered to the subject in various aspects is represented by characteristics of a post-administration solution, or an aqueous solution comprising the anti-EGFRvIII agent which is passed through the administration system, e.g., infusion system, collected and then analyzed in vitro. In exemplary aspects, the average cumulative subvisible particle counts/mL of the aqueous solution administered to the subject is less than about 15. The average cumulative subvisible particle counts/mL may be determined by using an HIAC™ liquid particle counter, such as an HIAC™ 8011+ or HIAC™ 9703+ liquid particle counter (Beckman Coulter, Indianapolis, IN). In various aspects, the subvisible particles are less than about 125 µm, for example, about 10 µm or about 25 µm.Optionally, the average cumulative subvisible particle counts/mL of the aqueous solution administered to the subject is less than about 10 (e.g., less than about 9, less than about 8, less than about 7, less than about 6, less than about 5, less than about 4, less than about 3, less than about 2, less than about 1). In exemplary aspects, the average cumulative subvisible particle counts/mL of the post-administration solution is less than 15, optionally, less than about 10 (e.g., less than about 9, less than about 8, less than about 7, less than about 6, less than about 5, less than about 4, less than about 3, less than about 2, less than about 1), as determined by HIAC. In exemplary aspects, the aqueous solution administered to the subject has less than 5 visible particles greater than or equal to 125 µm.Optionally, upon visual inspection, the post-administration solution has less than 5 visible particles greater than or equal to 125 µm. In various embodiments, the % high molecular weight (HMW) species of the aqueous solution administered to the subject is less than 5%. In various instances, the % high molecular weight (HMW) species of the aqueous solution administered to the subject is less than 2% and/or the % main peak of the anti-EGFRvIII agent is greater than 95%. Optionally, the % high molecular weight (HMW) species of the post-administration solution is less than 5%, as determined by SE-HPLC. In various instances, the % high molecular weight (HMW) species of the post-administration solution is less than 2% and/or the % main peak of the anti-EGFRvIII agent is greater than 95%, as determined by SE-HPLC. In exemplary aspects, greater than 95% of the anti-EGFRvIII agent in the aqueous solution before passing through the administration system (e.g., infusion system) is administered to the subject. In exemplary aspects, the % recovery of the anti-EGFRvIII agent in the post-administration solution is greater than 95% as determined by UV-VIS spectroscopy or RP-HPLC.

With regard to the presently disclosed methods and kits, the aqueous solution, in exemplary aspects, comprising the anti-EGFRvIII agent is suitable for administering to a subject via infusion. In various aspects, the aqueous solution is sterile and isotonic. In exemplary instances, the aqueous solution comprises saline, optionally, about 0.5% (v/v) to about 1.5% (v/v) saline. In various instances, the aqueous solution comprises about 0.5% (v/v) to about 1.4% (v/v) saline, about 0.5% (v/v) to about 1.3% (v/v) saline, about 0.5% (v/v) to about 1.2% (v/v) saline, about 0.5% (v/v) to about 1.1% (v/v) saline, about 0.5% (v/v) to about 1.0% (v/v) saline, about 0.5% (v/v) to about 0.9% (v/v) saline, about 0.5% (v/v) to about 0.8% (v/v) saline, about 0.5% (v/v) to about 0.7% (v/v) saline, about 0.5% (v/v) to about 0.6% (v/v) saline, about 0.6% (v/v) to about 1.5% (v/v) saline, about 0.7% (v/v) to about 1.5% (v/v) saline, about 0.8% (v/v) to about 1.5% (v/v) saline, about 0.9% (v/v) to about 1.5% (v/v) saline, about 1.0% (v/v) to about 1.5% (v/v) saline, about 1.1% (v/v) to about 1.5% (v/v) saline, about 1.2% (v/v) to about 1.5% (v/v) saline, about 1.3% (v/v) to about 1.5% (v/v) saline, or about 1.4% (v/v) to about 1.5% (v/v) saline.

In exemplary aspects, the aqueous solution comprises intravenous solution stabilizer (IVSS). IVSS is described in International Patent Application Publication No. WO2018/204907, which incorporated herein by reference. In various aspects, IVSS is a sterile, preservative-free, colorless to slightly yellow, clear solution comprising citric acid monohydrate, lysine hydrochloride, polysorbate 80, sodium hydroxide to adjust pH to 7.0, and water for injection. In exemplary aspects, IVSS comprises about 25 mM citric acid, about 1.25 M L-lysine HCI, about 0.1% (w/v) polysorbate 80, at pH 7.0. In various instances, the IVSS is provided as a single-dose in a vial comprising citric acid monohydrate (52.5 mg), lysine hydrochloride (2283.8 mg), polysorbate 80 (10 mg), sodium hydroxide to adjust pH to 7.0, and water for injection. In various aspects, the aqueous solution comprises about 2% (v/v) to about 6% (v/v) IVSS, optionally, about 4% (v/v) IVSS. In exemplary aspects, the aqueous solution comprises about 2% (v/v) to about 5.5% (v/v) IVSS, about 2% (v/v) to about 5.0% (v/v) IVSS, about 2% (v/v) to about 4.5% (v/v) IVSS, about 2% (v/v) to about 4.0% (v/v) IVSS, about 2% (v/v) to about 3.5% (v/v) IVSS, about 2% (v/v) to about 3.0% (v/v) IVSS, about 2% (v/v) to about 2.5% (v/v) IVSS, about 2.5% (v/v) to about 6% IVSS, about 3.0% (v/v) to about 6% IVSS, about 3.5% (v/v) to about 6% IVSS, about 4.0% (v/v) to about 6% IVSS, about 4.5% (v/v) to about 6% IVSS, about 5.0% (v/v) to about 6% IVSS, or about 5.5% (v/v) to about 6% IVSS. To facilitate the use of IVSS, it can be provided as prefilled vials for bedside mixing, such as about 10 mL/vial in prefilled vials. The target fill volume of each vial could be slightly higher (e.g., target fill volume at about 10.6 mL per vial) to account for the potential loss during clinical use.

In various aspects, the anti-EGFRvIII agent is present in the aqueous solution at a concentration of less than 75 ng/mL, optionally, less than 50 ng/mL. In some instances, the anti-EGFRvIII agent is present in the aqueous solution at a concentration of about 75 ng/mL to about 500 ng/mL, optionally, about 200 ng/mL to about 400 ng/mL. In exemplary aspects, the final volume of the aqueous solution is about 100 mL to about 300 mL solution. In exemplary instances, the anti-EGFRvIII agent is any one of those described herein. In certain embodiments, the anti-EGFRvIII agent described herein comprises the amino acid sequence of SEQ ID NO: 12. In certain embodiments, the anti-EGFRvIII agent described herein comprises the amino acid sequence of SEQ ID NO: 13.

In various instances, the anti-EGFRvIII agent is present in the aqueous solution at a concentration in the range from 0.5 mg/ml to 20 mg/ml, preferably 0.5 µg/ml to 10 or 5 mg/ml and the aqueous solution comprises at least one preservative selected from benzyl alcohol, chlorobutanol, meta-cresol, methylparaben, phenoxyethanol, propylparaben and thiomerosal at a concentration effective to inhibit the growth of microbes, and a diluent. Optionally, the aqueous solution comprises the anti-EGFRvIII agent is present at a concentration in the range selected from the group consisting of: (a) 0.5 to 200 g/ml at a pH of 6.5 to 7.5, or (b) 0.5 to 1000 g/ml at a pH of 4.0 to 6.0, or (c) 0.5 M9 to 2 mg in the presence of a CD3 binding domain stabilizing agent, preferably citrate, at a pH of 4.0 to 7.5 or (d) 0.5 mg to 20 mg, preferably 0.05 MQ/ml to 10 or 5 mg/ml, at a pH of 4.0 to 7.5, preferably 4.0 to 6.0. In various instances, the preservative is present in a concentration in the range from 0.001 to 1.0% (w/v). Optionally, the preservative is present in a concentration in the range from 0.009 to 0.9% (w/v), preferably 0.11 to 0.9% or 0.5 to 0.75% (w/v). The diluent in various aspects is a buffer comprising a salt selected from the group consisting of phosphate, acetate, citrate, succinate and tartrate, and/or wherein the buffer comprises histidine, glycine, TRIS glycine, Tris, or mixtures thereof. In exemplary instances, the diluent is a buffer selected from the group consisting of potassium phosphate, acetic acid/sodium acetate, citric acid/sodium citrate, succinic acid/sodium succinate, tartaric acid/sodium tartrate, and histidine/histidine HCI or mixtures thereof. Optionally, the diluent is a buffer present at a concentration in the range of 0.1 to 150 mM, preferably in the range of 0.25 to 50 mM. In various aspects, diluent is a buffer comprising citrate, optionally, at a concentration the range of 0.25 to 50 mM. In some aspects, the pH of the composition is in the range of 4.0 to 8.0, preferably in the range of pH 4.0 to 5.0 or 6.0 to 7.5, preferably at pH 7.0. In exemplary aspects, the aqueous solution further comprises one or more excipients selected from the group consisting of sucrose, trehalose, mannitol, sorbitol, arginine, lysine, polysorbate 20, polysorbate 80, poloxamer 188, pluronic and combinations thereof. Optionally, the aqueous solution comprises polysorbate, preferably polysorbate 80, and/or lysine HCL. In exemplary instances, the aqueous solution does not comprise polysorbate, preferably polysorbate 80, and/or lysine HCL if the concentration of the anti-EGFRvIII agent is at least 10, 15 or 20 µg/ml, preferably 15 µg/ml. In various aspects, the aqueous solution comprises at least 0.25 mM citrate, at least 0.0125 mM lysine and/or at least 0.001 % polysorbate 80 at a pH of 6.5 to 7.5. In various aspects, the anti-EGFRvIII agent is administered for at least 14 days at the initial dose of from about 15 µg/day to about 12000 µg/day. In various instances, the anti-EGFRvIII agent is administered for at least 14 days at the initial dose of from about 1500 µg/day to about 6000 µg/day. In exemplary instances, the anti-EGFRvIII agent is administered for at least 14 days at the initial dose of from about 3000 µg/day to about 6000 µg/day. In exemplary aspects, the anti-EGFRvIII agent is administered for at least 28 days at the initial dose of from about 15 µg/day to about 12000 µg/day. In various aspects, anti-EGFRvIII agent is administered for at least 28 days at the initial dose of from about 1500 µg/day to about 6000 µg/day. The anti-EGFRvIII agent, in various instances, is administered for at least 28 days at the initial dose of from about 3000 µg/day to about 6000 µg/day. In exemplary instances, the anti-EGFRvIII agent is administered at a 14-day on / 14-day off cycle, or a 28-day on / 14-day off cycle. The anti-EGFRvIII agent is, in exemplary aspects, administered to the subject at a dose of from 3000 µg/day to 6000 µg/day, at a 14-day on / 14-day off cycle, or a 28-day on / 14-day off cycle.

In exemplary embodiments of the present disclosure, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In various aspects, the subject has a neoplastic disease, e.g., any one of those described herein. The term “patient”, “subject”, or “mammal” as used herein refers to any “patient”, “subject”, or “mammal” including humans, cows, horses, dogs and cats. In one embodiment of the invention, the mammal is a human. In various instances, the subject is an human adult, e.g., aged 18 years or older.

In various aspects, the subject has a neoplastic disease and the method treats the neoplastic disease. As used herein, the term “neoplastic disease” refers to any condition that causes growth of a tumor. In exemplary aspects, the tumor is a benign tumor. In exemplary aspects, the tumor is a malignant tumor. In various aspects, the neoplastic disease is cancer. The cancer in various aspects is acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, or urinary bladder cancer. In particular aspects, the cancer is head and neck cancer, ovarian cancer, cervical cancer, bladder cancer, oesophageal cancer, pancreatic cancer, gastrointestinal cancer, gastric cancer, breast cancer, endometrial cancer, colorectal cancer, hepatocellular carcinoma, glioblastoma, bladder cancer, lung cancer, e.g., non-small cell lung cancer (NSCLC), or bronchioloalveolar carcinoma. In particular embodiments, the tumor is non-small cell lung cancer (NSCLC), head and neck cancer, renal cancer, triple negative breast cancer, or gastric cancer. In exemplary aspects, the subject has a tumor (e.g., a solid tumor, a hematological malignancy, or a lymphoid malignancy) and the pharmaceutical composition is administered to the subject in an amount effective to treat the tumor in the subject. In other exemplary aspects, the tumor is non-small cell lung cancer (NSCLC), small cell lung cancer (SCLC), head and neck cancer, renal cancer, breast cancer, melanoma, ovarian cancer, liver cancer, pancreatic cancer, colon cancer, prostate cancer, gastric cancer, lymphoma or leukemia, and the pharmaceutical composition is administered to the subject in an amount effective to treat the tumor in the subject.

The terms “cancer” and “cancerous” when used herein refer to or describe the physiological condition in mammals that is typically characterized by unregulated cell growth. Examples of cancer include, without limitation, carcinoma, lymphoma, sarcoma, blastoma and leukemia. More particular examples of such cancers include squamous cell carcinoma, lung cancer, pancreatic cancer, cervical cancer, bladder cancer, hepatoma, breast cancer, colon carcinoma, and head and neck cancer, ovarian cancer, and endometrial cancer.

In various aspects, the neoplastic disease is a cancer or a tumor. Optionally, the cancer or tumor is an EGFRvIII-positive cancer or tumor. In exemplary instances, the cancer is a squamous cell tumor, such as non-small cell lung cancer (NSCLC). In exemplary aspects, the cancer is glioblastoma or malignant glioma.

The term “treatment” includes prophylactic and/or therapeutic treatments. If it is administered prior to clinical manifestation of a condition, the treatment is considered prophylactic. Therapeutic treatment includes, e.g., ameliorating or reducing the severity of a disease, or shortening the length of the disease. The term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating a neoplastic disease of the present disclosure can provide any amount or any level of treatment. Furthermore, the treatment provided by the methods of the present disclosure can include treatment of one or more conditions or symptoms or signs of the neoplastic disease being treated. Also, the treatment provided by the methods of the present disclosure can encompass slowing the progression of the neoplastic disease. For example, the methods can treat neoplastic disease by virtue of enhancing the T cell activity or an immune response against the neoplastic disease, reducing tumor or cancer growth or tumor burden, reducing metastasis of tumor cells, increasing cell death of tumor or cancer cells or increasing tumor regression, and the like. In accordance with the foregoing, provided herein are methods of reducing tumor growth or tumor burden or increasing tumor regression in a subject. In exemplary embodiments, the method comprises administering to the subject an anti-EGFRvIII agent. The terms “treat”, “treating” and “treatment” as used herein refer to therapy, including without limitation, curative therapy, prophylactic therapy, and preventative therapy. Prophylactic treatment generally constitutes either preventing the onset of disorders altogether or delaying the onset of a pre-clinically evident stage of disorders in individuals.

In various aspects, the methods treat by way of delaying the onset or recurrence of the neoplastic disease by at least 1 day, 2 days, 4 days, 6 days, 8 days, 10 days, 15 days, 30 days, two months, 3 months, 4 months, 6 months, 1 year, 2 years, 3 years, 4 years, or more. In various aspects, the methods treat by way increasing the survival of the subject. In exemplary aspects, the methods of the present disclosure provide treatment by way of delaying the occurrence or onset of metastasis. In various instances, the methods provide treatment by way of delaying the occurrence or onset of a new metastasis. Accordingly, provided herein are methods of delaying the occurrence or onset of metastasis in a subject with cancer. In exemplary embodiments, the method comprises administering an anti-EGFRvIII agent to the subject.

In exemplary instances, the treatment provided may be described in terms of or supported by data obtained from a clinical trial wherein the endpoints of the trial are progression-free survival (PFS), overall survival (OS), or time to deterioration of Eastern Cooperative Oncology Group (ECOG) performance status. In various aspects, the present disclosure provides a method of increasing PFS, OS, or time to deterioration of ECOG performance status in a subject with a neoplastic disease. As used herein, the term “progression-free survival” or “PFS” means the time a treated patient experiences without cancer getting worse (by whatever measure is being used to measure worsening). The term “overall survival” means how long the patient lives after treatment. ECOG performance status is a grade or score according to a scale used by doctors and researchers to assess a patient’s disease, e.g., how the disease is progressing/regressing, how the disease affects the daily living abilities of the patient, and determine appropriate treatment and prognosis. ECOG performance status is determined according to the following criteria:

| SCORE | ECOG |
|---|---|
| 0 | Fully active, able to carry on all pre-disease performance without restriction |
| 1 | Restricted in physically strenuous activity but ambulatory and able to carry out work of a light or sedentary nature, e.g., light house work, office work |
| 2 | Ambulatory and capable of all selfcare but unable to carry out any work activities. Up and about more than 50% of waking hours |
| 3 | Capable of only limited selfcare, confined to bed or chair more than 50% of waking hours |
| 4 | Completely disabled. Cannot carry on any selfcare. Totally confined to bed or chair |
| 5 | Dead |

Oken et al., Am. J. Clin. Oncol 5: 649-655 (1982)

### 5. Kits and Articles of Manufacture

The present disclosure provides kits. In exemplary embodiments, the kit comprises an administration system and an anti-EGFRvIII agent, wherein one or more of the components of the administration system, or parts thereof, which contact the aqueous solution substantially lack a PVC or PVC plasticizer and/or substantially lacks air. In various aspects, the administration system is any of those described herein, including by not limited to, an infusion system comprising an infusion line. In various aspects, one or more of the components of the administration system (e.g., infusion system), or parts thereof, which contact the aqueous solution substantially lack(s) PVC or PVC plasticizer. In various aspects, the one or more parts comprise(s) a polymer other than PVC, e.g., plasticized PVC. In exemplary instances, the one or more parts comprise(s) polyolefin elastomer (POE) or polyurethane (PUR) or ethylene vinyl acetate (EVA). Optionally, the one or more parts is composed entirely of the POE, PUR, or EVA. Alternatively, the one or more parts comprise(s) PVC, e.g., PVC with a plasticizer, yet the portion(s) which contact(s) the aqueous solution are coated with a material other than PVC. Optionally, the one or more portions which contact(s) the aqueous solution are coated with POE, PUR, or EVA. In various aspects, the infusion line or parts thereof that contact the aqueous solution substantially lack PVC or a PVC plasticizer. In various aspects, the infusion line comprises a polymer other than PVC. In exemplary instances, the infusion line comprises POE and/or PUR and/or EVA. In various aspects, the infusion line is composed entirely of the polymer other than PVC, e.g., the infusion line is composed entirely POE, PUR, or EVA. Alternatively, the infusion line comprises PVC yet the portion(s) of the infusion line which contact(s) the aqueous solution is/are coated with a material other than PVC, e.g., coated with POE, PUR, or EVA. Optionally, the one or more portions which contact(s) the aqueous solution, e.g., the interior face of the infusion line, are coated with POE or PUR or EVA. Optionally, the administration system comprises a container for containing the aqueous solution and at least part of the container substantially lacks PVC or a PVC plasticizer.

In various embodiments, the administration system comprises a container for containing the aqueous solution and when the container comprises the aqueous solution at least part of the container substantially lacks air. In various instances, less than about 5% of the volume of the container is air, when the container comprises the aqueous solution. Optionally, less than about 4%, less than about 3%, less than about 2%, or less than about 1% of the volume of the container is air, when the container comprises the aqueous solution. In various aspects, at least part of the IV bag substantially lacks PVC or a PVC plasticizer, optionally, wherein the part of the IV bag that contacts the aqueous solution substantially lacks PVC or a PVC plasticizer. In some instances, at least part of the IV bag comprises an ethylene vinyl acetate (EVA) or a polyolefin elastomer (POE). For instance, the IV bag is composed entirely of the EVA or POE. In exemplary aspects, the method comprises administering to the subject an aqueous solution comprising an anti-EGFRvIII agent using an infusion system comprising an IV bag. In some aspects, when the IV bag comprises the aqueous solution, less than about 5% of the volume of the container is air. Optionally, less than about 4%, less than about 3%, less than about 2%, or less than about 1% of the volume of the IV bag is air, when the IV bag comprises the aqueous solution. In various aspects, at least part of the IV bag substantially lacks PVC or a PVC plasticizer, optionally, wherein the part of the IV bag that contacts the aqueous solution substantially lacks PVC or a PVC plasticizer. In some instances, at least part of the IV bag comprises an ethylene vinyl acetate (EVA) or a polyolefin elastomer (POE). For instance, the IV bag is composed entirely of the EVA or POE.

In various embodiments, the aqueous solution, in exemplary aspects, comprising the anti-EGFRvIII agent comprises saline, optionally, about 0.5% to about 1.5% (v/v) saline. In various instances, the aqueous solution comprises about 0.5% to about 1.4% (v/v) saline, about 0.5% to about 1.3% (v/v) saline, about 0.5% to about 1.2% (v/v) saline, about 0.5% to about 1.1% (v/v) saline, about 0.5% to about 1.0% (v/v) saline, about 0.5% to about 0.9% (v/v) saline, about 0.5% to about 0.8% (v/v) saline, about 0.5% to about 0.7% (v/v) saline, about 0.5% to about 0.6% (v/v) saline, about 0.6% to about 1.5% (v/v) saline, about 0.7% to about 1.5% (v/v) saline, about 0.8% to about 1.5% (v/v) saline, about 0.9% to about 1.5% (v/v) saline, about 1.0% to about 1.5% (v/v) saline, about 1.1% to about 1.5% (v/v) saline, about 1.2% to about 1.5% (v/v) saline, about 1.3% to about 1.5% (v/v) saline, or about 1.4% to about 1.5% (v/v) saline. Optionally, the aqueous solution comprises about 2% (v/v) to about 6% (v/v) intravenous saline solution (IVSS), optionally, about 4% (v/v) IVSS. In exemplary aspects, the aqueous solution comprises about 2% (v/v) to about 5.5% (v/v) IVSS, about 2% (v/v) to about 5.0% (v/v) IVSS, about 2% (v/v) to about 4.5% (v/v) IVSS, about 2% (v/v) to about 4.0% (v/v) IVSS, about 2% (v/v) to about 3.5% (v/v) IVSS, about 2% (v/v) to about 3.0% (v/v) IVSS, about 2% (v/v) to about 2.5% (v/v) IVSS, about 2.5% (v/v) to about 6% IVSS, about 3.0% (v/v) to about 6% IVSS, about 3.5% (v/v) to about 6% IVSS, about 4.0% (v/v) to about 6% IVSS, about 4.5% (v/v) to about 6% IVSS, about 5.0% (v/v) to about 6% IVSS, or about 5.5% (v/v) to about 6% IVSS. In various aspects, the anti-EGFRvIII agent is present in the aqueous solution at a concentration of less than 75 ng/mL, optionally, less than 50 ng/mL. In some instances, the anti-EGFRvIII agent is present in the aqueous solution at a concentration of about 75 ng/mL to about 500 ng/mL, optionally, about 200 ng/mL to about 400 ng/mL. In exemplary aspects, the final volume of the aqueous solution is about 100 mL to about 300 mL solution. In exemplary instances, the anti-EGFRvIII agent is any one of those described herein. In certain embodiments, the anti-EGFRvIII agent described herein comprises the amino acid sequence of SEQ ID NO: 12. In certain embodiments, the anti-EGFRvIII agent described herein comprises the amino acid sequence of SEQ ID NO: 13.

The present disclosure provides articles of manufacture. In exemplary embodiments, the article of manufacture comprises a container containing an aqueous solution comprising an anti-EGFRvIII agent, wherein the container substantially lacks air. In various instances, less than about 5% of the volume of the container is air, when the container comprises the aqueous solution. Optionally, less than about 4%, less than about 3%, less than about 2%, or less than about 1% of the volume of the container is air. In various aspects, the container is an IV bag and at least part of the IV bag substantially lacks PVC or a PVC plasticizer. In some instances, at least part of the IV bag comprises an ethylene vinyl acetate (EVA) or a polyolefin elastomer (POE). For instance, the IV bag is composed entirely of the EVA or POE, or alternatively, the interior of the IV bag is lined or coated with EVA or POE. In exemplary instances, the aqueous solution, in exemplary aspects, comprising the anti-EGFRvIII agent comprises saline, optionally, about 0.5% to about 1.5% (v/v) saline. In various instances, the aqueous solution comprises about 0.5% to about 1.4% (v/v) saline, about 0.5% to about 1.3% (v/v) saline, about 0.5% to about 1.2% (v/v) saline, about 0.5% to about 1.1% (v/v) saline, about 0.5% to about 1.0% (v/v) saline, about 0.5% to about 0.9% (v/v) saline, about 0.5% to about 0.8% (v/v) saline, about 0.5% to about 0.7% (v/v) saline, about 0.5% to about 0.6% (v/v) saline, about 0.6% to about 1.5% (v/v) saline, about 0.7% to about 1.5% (v/v) saline, about 0.8% to about 1.5% (v/v) saline, about 0.9% to about 1.5% (v/v) saline, about 1.0% to about 1.5% (v/v) saline, about 1.1% to about 1.5% (v/v) saline, about 1.2% to about 1.5% (v/v) saline, about 1.3% to about 1.5% (v/v) saline, or about 1.4% to about 1.5% (v/v) saline. Optionally, the aqueous solution comprises about 2% (v/v) to about 6% (v/v) intravenous saline solution (IVSS), optionally, about 4% (v/v) IVSS. In exemplary aspects, the aqueous solution comprises about 2% (v/v) to about 5.5% (v/v) IVSS, about 2% (v/v) to about 5.0% (v/v) IVSS, about 2% (v/v) to about 4.5% (v/v) IVSS, about 2% (v/v) to about 4.0% (v/v) IVSS, about 2% (v/v) to about 3.5% (v/v) IVSS, about 2% (v/v) to about 3.0% (v/v) IVSS, about 2% (v/v) to about 2.5% (v/v) IVSS, about 2.5% (v/v) to about 6% IVSS, about 3.0% (v/v) to about 6% IVSS, about 3.5% (v/v) to about 6% IVSS, about 4.0% (v/v) to about 6% IVSS, about 4.5% (v/v) to about 6% IVSS, about 5.0% (v/v) to about 6% IVSS, or about 5.5% (v/v) to about 6% IVSS. In various aspects, the anti-EGFRvIII agent is present in the aqueous solution at a concentration of less than 75 ng/mL, optionally, less than 50 ng/mL. In some instances, the anti-EGFRvIII agent is present in the aqueous solution at a concentration of about 75 ng/mL to about 500 ng/mL, optionally, about 200 ng/mL to about 400 ng/mL. In exemplary aspects, the final volume of the aqueous solution is about 100 mL to about 300 mL solution. In exemplary instances, the anti-EGFRvIII agent is any one of those described herein. In certain embodiments, the anti-EGFRvIII agent described herein comprises the amino acid sequence of SEQ ID NO: 12. In certain embodiments, the anti-EGFRvIII agent described herein comprises the amino acid sequence of SEQ ID NO: 13.

In exemplary aspects, the kit of the present disclosure comprises an IV bag pre-filled with an aqueous solution comprising saline and/or IVSS and a container comprising an anti-EGFRvIII agent. Optionally, the anti-EGFRvIII agent is lyophilized. In alternative aspects, the container comprises the anti-EGFRvIII agent in an aqueous solution comprising saline and/or IVSS.

The following examples are given merely to illustrate the present invention and not in any way to limit its scope.

### EXAMPLES

The following example provide an evaluation of AMG 596 infusion solution held in IV bags and then pumped through infusion lines. The examples show that when AMG 596 is pumped through a polyvinyl chloride (PVC) infusion line the molecule particulates, revealing an incompatibility with PVC infusion lines. However, when either polyethylene (PE) lined or polyurethane (PUR) lined infusion lines were subsequently tested for compatibility with AMG 596, the particulation of the protein was not observed. Additionally, no incompatibility was observed as a result of product contact with IV bags composed of ethylene vinyl acetate (EVA) or polyolefins. For all combinations of IV bags and infusion lines tested all the quality attributes assessed were at acceptable levels indicating compatibility, with the exception of visible and sub-visible particle levels. When an infusion line composed of PVC was used in the compatibility assessment, the solution collected post-infusion, but not pre-infusion, was very cloudy and contained unacceptable levels of visible particles indicating protein aggregation. Because the solution collected directly from the IV bags, pre-infusion, was clear, the action of being pumped through an infusion line composed of PVC was definitively the cause of protein aggregation and a cloudy infusion solution. However, if the PVC infusion line was replaced with either a PE or PUR infusion line, the visible particle generation was not observed, and all other quality attributes assessed indicated that the AMG 596 infusion solution was compatible with these lines. Thus, the PVC lines, specifically, were identified as incompatible clVi components for infusion of AMG 596.

### Example 1

This example describes short-term studies of infusion solutions of AMG596 in IV bags.

AMG 596 drug product (DP) is admixed in an IV bag with saline and an intravenous solution stabilizer (IVSS) to make an infusion solution. This solution is purposed for infusing into a patient by being pumped with an infusion pump through an infusion line into the patient. An initial assessment of the AMG 596 infusion solution held in an IV bag (250 mL EVA IV bag, MediBag, Beavercreek, OH) and then pumped through an infusion line using a CADD administration set (100 mL) was carried out. In this study, the infusion solution comprised saline, AMG 596 (either 33 µg/mL or 330 µg/mL) with or without ± 4% (v/v) IVSS. The final volume in the IV bag was 100 mL. The bag was agitated at 60 rpm for 48 hours at 25° C. The infusion solution in the IV bag was then evaluated for particle count by HIAC, change in % oligomers and/or fragments by SEC, and protein recovery by UV-Vis spectroscopy.

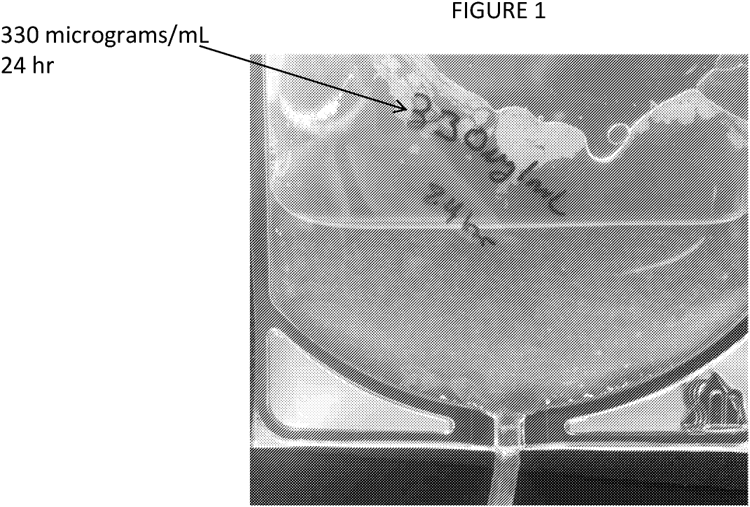

FIG. 1 shows the results of the initial assessment. As shown in this figure, the infusion solution in the IV bag showed heavy particulation after agitation. The concentration of the AMG 596 had dropped by 67% of the initial concentration at the 24-hour time point.

Given the loss of protein and the heavy particulation, alternative IV bags were assessed. In this study, five additional IV bags comprising EVA or POE were evaluated, alongside the MediBag IV bag used in the initial assessment. Among the IV bags evaluated were the Exactamix EVA IV bag (250 mL, Baxter Healthcare Corp., Deerfield, IL), the Secure EVA IV bag (50 mL, Medisca, Quebec, Canada), the ASI EVA IV bag (100 mL, Advanced Scientifics, Inc., Millersburg, PA), the B Braun Excel POE IV bag (250 mL, B. Braun Medical, Inc., Bethlehem, PA), and the B Braun PAB^{®} POE IV bag (150 mL, B. Braun Medical, Inc., Bethlehem, PA). Each bag was filled with a total volume of 100 mL of the infusion solution comprising AMG 596 (either 33 µg/mL or 330 µg/mL) in IVSS and saline with or without air. The bag was agitated at 60 rpm for 48 hours at 25° C. The infusion solution in the IV bag was then evaluated for particle count by HIAC, change in % oligomers and/or fragments by SEC, and protein recovery by UV-Vis spectroscopy.

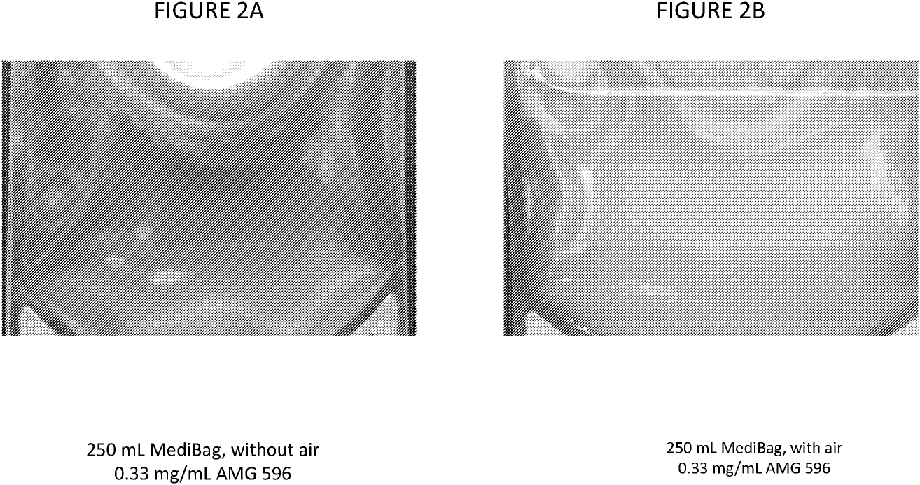

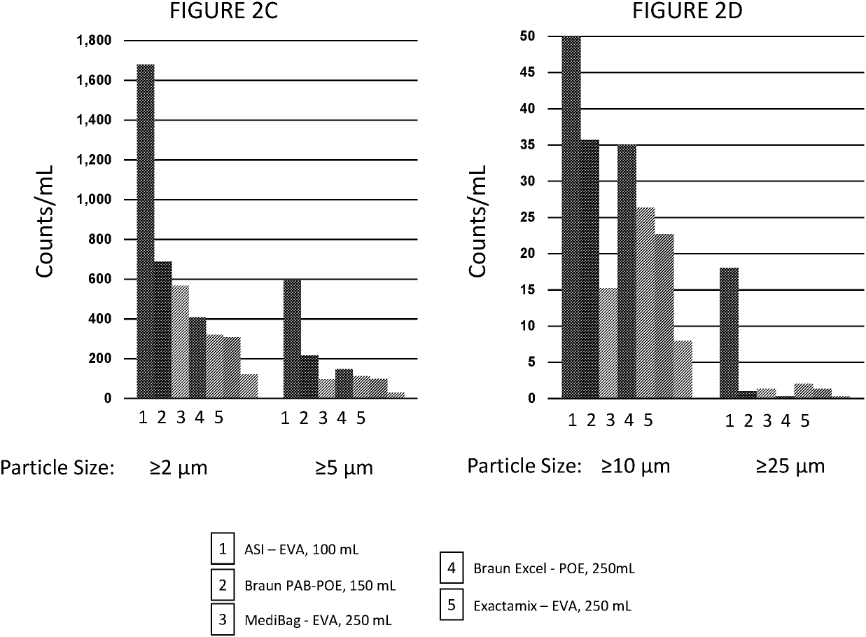

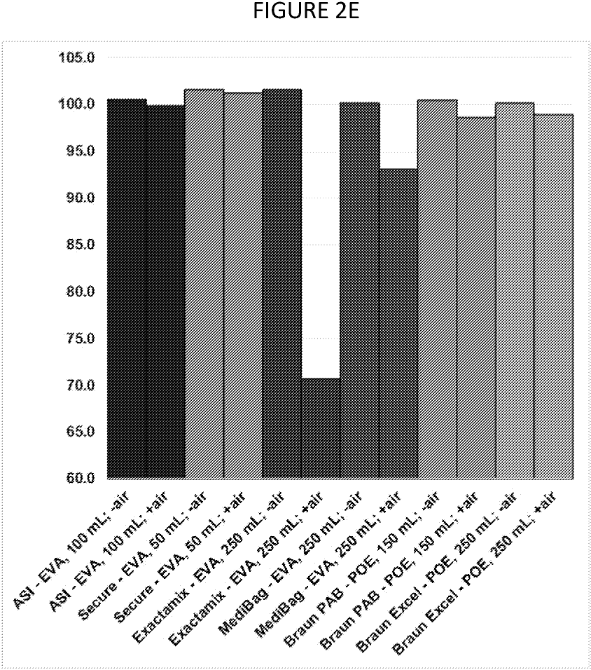

Exemplary results are shown in FIGS. 2A and 2B. As shown in these figures, the infusion solution in the IV bag with air consistently showed opalescence and heavy particulation post-agitation, compared to the infusion solution in the IV bag without air. Particle counts were measured by HIAC for particles of different sizes. FIG. 2C shows the particle counts/mL for particles sized greater than or equal to 2 µm and greater than or equal to 5 µm, while FIG. 2D, shows the particle counts/mL for particles sized greater than or equal to 10 µm and greater than or equal to 25 µm. These results show that the ASI IV bag (red bars in FIGS. 2C and 2D) had significantly higher particle counts than the other IV bags. The MediBag had the lowest number of ≥10 µm particles and a low number of ≥25 µm particles. Protein recovery was measured and there was 100% recovery provided that air was purged from the IV bag (FIG. 2E). Size exclusion chromatography was carried out and demonstrated that the presence of absence of air did not change the levels of dimer after 24 hours of agitation at 60 rpm 25° C. in any of the IV bags tested. For the Exactamix IV bag, an extra “leachable” peak was present in the small molecule retention time of the chromatogram.

In a subsequent experiment, an inline filter was placed between the solution set and catheter to determine if the filter could adsorb AMG 596. An infusion solution comprising AMG 596 (either 33 µg/mL or 330 µg/mL) was pumped through an infusion line using a CADD cassette (100 mL) with a PES filter and protein recovery was measured. There was no detectable loss of AMG 596 using the inline filter.

These results suggest that AMG 596 infusion solutions are compatible with IV bags composed of ethylene vinyl acetate (EVA) or polyolefins (POE).

### Example 2

This example describes the materials and methods used to carry out the experiments of Example 1.

Materials - The following materials were used: CADD^{®} administration set (274 cm (108 in.), 3.3 mL) (Smiths Medical, St. Paul, MN) comprising a 0.2 µm air-eliminating filter and containing Trioctyl Trimellitat (TOTM) and < 0.2% DEHP; EVA IV Bags, 100-300 mL, phthalate-free (e.g., DEHP-free) (ICU Medical, San Clemente, CA); CADD infusion pump; mPolyolefin IV Bags (150 mL), B Braun, PAB Partial Additive Bag, S5904-52; Intravia IV Bags, 500 mL (p/n 2J8003); Sapphire primary polyethylene lined clear infusion set (p/n 16422-01); QCore Medical, Sapphire Infusion pump (ref# 15031-000-0028); Saline: Hospira, NDC: 0409-4888-06; Filter: B Braun, Supor membrane 0.2 m air eliminating filter, p/n 473036.

The following methods were carried out:

### Chemical Modification of the Molecule by Cation Exchange (CEX) Chromatography

This technique was carried out to determine the purity and distribution of charge variants of AMG 596. Charge variants of AMG 596 were separated by cation exchange chromatography using a cation exchange analytical column and then eluted from the column using a mobile phase gradient of increasing ionic strength. Proteins with less positive surface charge eluted earlier than proteins with a greater positive surface charge. Eluted charge variants were detected by UV detection, integrated, and the results for Main, Acidic, and Basic peaks were reported as the percentage of the total peak area.

### Oligomerization or Fragmentation by Size Exclusion Chromatography (SEC)

This method was carried out to measure the purity of AMG 596. SE-UHPLC separates proteins in solution based on their hydrodynamic volume using a size exclusion ultra-high performance analytical column. The high molecular weight (aggregate peaks) eluted earlier than the monomeric and lower molecular weight peaks. Components were eluted isocratically, detected by UV detection, integrated, and the results were reported as relative peak area percentages of high molecular weight, Main (monomer), and low molecular weight peaks, if any.

### pH of the Solution

The pH of samples was determined using a calibrated pH meter.

### Sub-Visible Particle Levels by Light Obscuration (HIAC)

This procedure determines the number of ≥ 10 µm and ≥ 25 µm particles per container. This test was performed to enumerate subvisible particles within specific size ranges. The apparatus used was an electronic, liquid-borne particle counting system that uses a light-obscuration sensor with a suitable sample-feeding device. Four aliquots (not less than one (1) mL each) from a pool solution with a total volume of not less than five (5) mL were degassed via vacuum and analyzed; data from the first aliquot was discarded and the number of particulates per container was calculated from the average of the remaining 3 measurements. Results were reported as the number of particles per container for particle sizes ≥ 10 µm and ≥ 25 µm. Additionally, particles ≥ 2 µm and ≥ 5 µm in size per container were monitored.

### Visible Particle Levels by Visual Inspection

The sample was visually inspected using a light to illuminate the sample inside a visual inspection booth. The number of particles having a size ≥ 125 µm was recorded.

### Protein Recovery Assay: UV-Vis Spectrophotometry

For the 0.2 mg/mL AMG 596 IV bags, the protein concentration of AMG 596 samples was determined using UV-Visible Light Spectroscopy. The absorbance of the sample was measured and the protein concentration was calculated using the following equation: [(A₂₈₀ - A₃₅₀)/2.0] (the extinction coefficient of AMG 596 is 2.0 mg⁻¹*cm⁻¹*mL).

### Protein Recovery Assay: Reversed Phase Titer Method

For the 90 ng/mL IV bags, the protein concentration of AMG 596 samples that have a concentration below the limit of detection of UV-Visible Light Spectroscopy was determined using reversed phase chromatography. The separation of AMG 596 from other impurities was achieved using a C8 column. AMG 596 was eluted from the column using a mixture of aqueous and organic solvents. The protein concentration was determined by measuring the area of the peak of the eluted AMG 596 and a standard curve and linear regression analysis to calculate the concentration of the sample.

### Example 3

This example describes longer-term studies of infusion solutions of AMG596 in IV bags.

The assessment was set up to mimic how the product would be prepared in a clinic and administered to a patient. IV bags were prepared with a solution composed of 4% (v/v) IVSS, 0.9% (v/v) saline, and AMG 596 drug product (DP) at either 0.2 mg/mL or 90 ng/mL for a final volume of either 126 mL or 150 mL, respectively. The IV bags were then held at 2-8° C. for ten days to cover a maximum hold time of the prepared IV bags for seven days. Next, the IV bags were placed on a shaker (60 rpm) at a controlled temperature of 30° C. for five days. This part of the experiment is to simulate the IV bag being carried on a patient for five days, to cover a maximum continuous administration time of four days. Finally, the AMG 596 infusion solution is pumped through an infusion line and collected to simulate the infusion of the solution into a patient.

Samples of the infusion solution were taken at the following times: after admixture in the IV bag (t0), after ten days held at 2-8° C., after five additional days following continual agitation at 30° C. both directly from the IV bags (pre-infusion) and after being pumped through the infusion lines (post-infusion). Samples taken at t=0 were obtained directly from the IV bag with a syringe. Fifteen-day samples were pumped through an inline filter containing a PVC infusion line with inline filter and using a CADD pump at either 5 mL/hr or 125 mL/hr. Control samples comprised saline and the IVSS without any protein.

These samples were analyzed to assess attributes to determine the quality and stability of the AMG 596 protein. The attributes analyzed were: chemical modification of the molecule by cation exchange (CEX) chromatography, oligomerization or fragmentation by size exclusion chromatography (SEC), pH of the solution, sub-visible particle levels by light obscuration (HIAC), visible particle levels by visual inspection, and recovery of the protein by either UV-Vis spectrophotometry for the 0.2 mg/mL AMG 596 IV bags or by a reversed phase titer method for the 90 ng/mL IV bags.

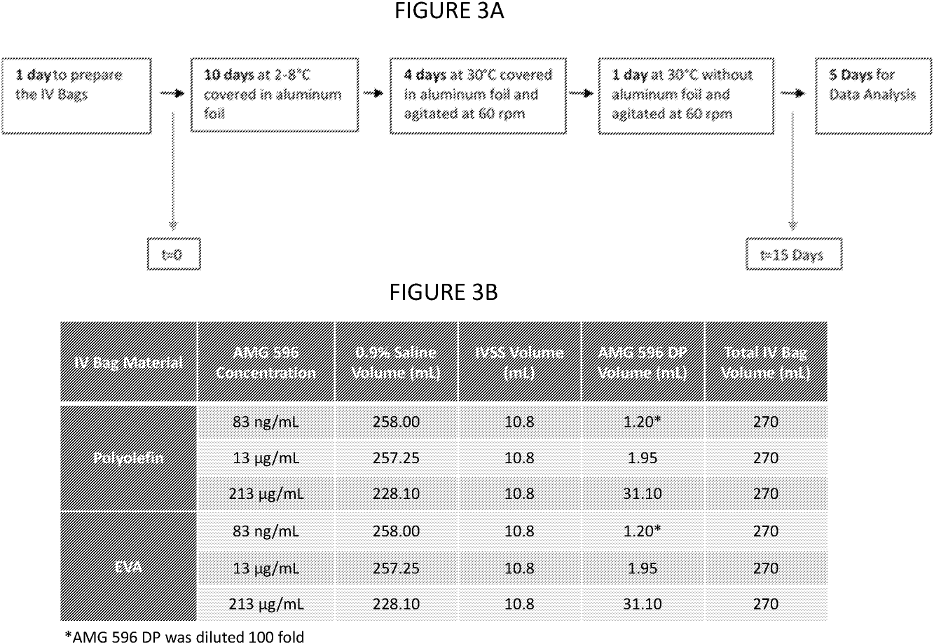

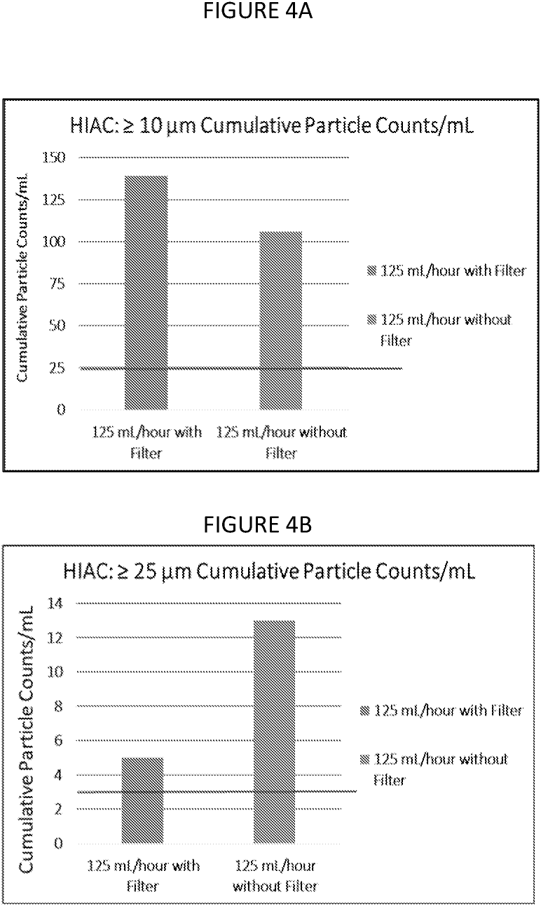

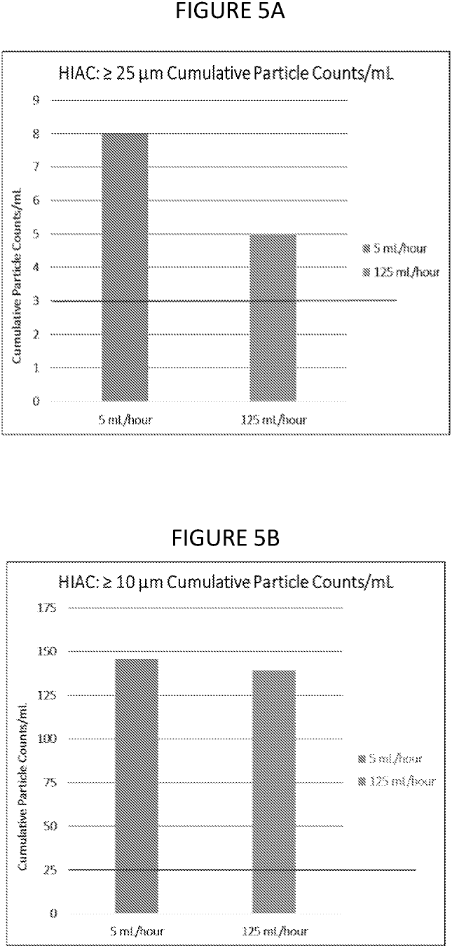

A summary of the experiment is shown in FIG. 3A and a summary of the details of the preparation of the IV bags at Day 1 is summarized in FIG. 3B.

Whereas the control samples were as clear as water, and the t=0 samples comprising 0.2 mg/mL AMG 256 were clear, the samples comprising 0.2 m/mL AMG 256 contained significant amounts of visible particles. Also, the fifteen-day samples comprising 90 ng/mL AMG 256 were slightly cloudy.

To assess the effects of just the infusion line, the above experiment was repeated but without an inline filter. The results are shown in FIGS. 4A and 4B. As shown in these figures, removing the filter had no effect on particulation, suggesting that the infusion solution is not compatible with the PVC infusion line.

To assess the effects of infusion speed, the above experiment was carried out at an infusion speed of either 5 mL per hour or 125 mL per hour. The results are shown in FIGS. 5A and 5B. As shown in these figures, infusion speed had no effect on particulation.

These results support that when AMG 596 is pumped through a polyvinyl chloride (PVC) infusion line the molecule particulates, revealing an incompatibility with PVC infusion lines. Visible particles were present with or without an inline filter and sub-visible particles were higher than acceptable levels with or without an inline filter. It is noted that the protein was not adsorbed to a significant level to the IV bags, infusion lines, or inline filter. The Percent Recovery of AMG 596 was within acceptable limits as determined by Biacore, Reversed Phase Chromatography, or UV-Visible Spectrophotometry. Also, infusion of the infusion solution through the infusion system did not lead to increased levels of soluble HMW species as measured by SE-UPLC.

### Example 4

This example demonstrates a study to test the compatibility of AMG 256 infusion solutions with non-PVC infusion lines and an inline filter.

This study was carried out as essentially described in Example 3 except that infusion lines lined with polyurethane (PUR) and comprising a 0.2 µm polyethersulfone (PES) filter was used.

Percent recovery and SE-HPLC results are shown in Tables 1 and 2, respectively.

TABLE 1

| IV Bag Material | AMG 596 Concentration | % Recovery after 15 Days | Analytical Method |
|---|---|---|---|
| EVA | 86 ng/mL | 108.8% | RP-HPLC |
| EVA | 200 µg/mL | 103.6% | UV-Visible Spectroscopy |

TABLE 2

| IV Bag Material | AMG 596 Concentration | % HMW t=0 | % HMW t=15 days | % Main Peak t= 0 | % Main Peak t=15 days |
|---|---|---|---|---|---|
| EVA | 200 ng/mL | 0.5% | 0.6% | 99.5% | 99.4% |

No visible particles (greater than or equal to 125 µm) were observed during visual inspections. Subvisible particle analysis by HIAC was carried out and revealed very low particle counts (Table 3).

TABLE 3

| IV Bag Material | AMG 596 Concentration | 10 µm Avg Cumulative Counts/mL | | 25 µm Avg Cumulative Counts/mL | |
|---|---|---|---|---|---|
| | | t=0 | t=15 days | t= 0 | t= 15 days |
| EVA | 86 ng/mL | 5.0 | 5.0 | 0.0 | 0.0 |
| EVA | 200 ng/mL | 6.0 | 11.0 | 0.5 | 0.5 |

These results suggest that use of either polyethylene (PE) lined or polyurethane (PUR) lined infusion lines in an infusion system for infusion solutions comprising AMG 596 does not cause particulation of the protein.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms including the indicated component(s) but not excluding other elements (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of treating an EGFRvIII-positive cancer or tumor in a subject, comprising administering to the subject an aqueous solution comprising an anti-EGFRvIII agent using an infusion system comprising an infusion line, wherein the infusion line substantially lacks polyvinyl chloride (PVC) or PVC plasticizer, and wherein said anti-EGFRvIII agent is a bispecific antigen-binding polypeptide comprising: a first binding domain that binds to human EGFRvIII and macaque EGFRvIII, and a second binding domain that binds to human CD3.

2. The method of claim 1, wherein the infusion line comprises a polyolefin elastomer (POE), polyurethane (PUR), or ethylene vinyl acetate (EVA).

3. The method of claim 1, wherein the average cumulative subvisible particle counts/mL post-infusion solution is less than 15, as determined by a liquid particle counter.

4. The method of claim 3, wherein the average cumulative subvisible particle counts/mL post-infusion solution is less than 10 as determined by a liquid particle counter.

5. The method of claim 1, wherein, upon visual inspection, the post-infusion solution has less than 5 visible particles greater than or equal to 125 µm.

6. The method of claim 1, wherein the % high molecular weight (HMW) species of the post-infusion solution is less than 5%, as determined by SE-HPLC.

7. The method of claim 1, wherein the % HMW species of the post-infusion solution is less than 2% and/or the % main peak of the anti-EGFRvIII agent is greater than 95%, as determined by SE-HPLC.

8. The method of claim 1, wherein the % recovery of the anti-EGFRvIII agent in the post-infusion solution is greater than 95% as determined by UV-VIS spectroscopy or RP-HPLC.

9. The method of claim 1, wherein the aqueous solution is administered to the subject by continuous intravenous infusion (cIVi).

10. The method of claim 1, wherein the anti-EGFRvIII agent is administered to the subject at a dose of from 3000 µg/day to 6000 µg/day, at a 14-day on / 14-day off cycle, or a 28-day on / 14-day off cycle.

11. The method of claim 1, wherein the infusion system further comprises an intravenous (IV) bag, an infusion pump, an infusion line filter, or a combination thereof.

12. The method of claim 11, wherein the infusion line filter is a polyethylsulfone (PES) filter.

13. The method of claim 11, wherein the IV bag comprises ethylene vinyl acetate (EVA) or a polyolefin elastomer (POE).

14. The method of claim 11, wherein the IV bag comprises the aqueous solution comprising the anti-EGFRvIII agent and air, wherein less than 5% of the total volume of the IV bag is air.

15. The method of claim 11, wherein the infusion pump is a CADD pump.

16. The method of claim 11, wherein the infusion pump is programmable, lockable, non-elastomeric, and/or has an alarm.

17. The method of claim 1, wherein the aqueous solution is infused at an infusion speed of about 1.5 ml/hour to about 150 mL/hour.

18. The method of claim 1, wherein said cancer is glioblastoma or malignant glioma.

19. The method of claim 1, wherein the aqueous solution comprises about 0.5% to about 1.5% (v/v) saline.

20. The method of claim 1, wherein the solution comprises about 2% (v/v) to about 6% (v/v) intravenous saline solution (IVSS).

21. The method of claim 1, wherein the anti-EGFRvIII agent is present in the aqueous solution at a concentration of less than 75 ng/mL.

22. The method of claim 21, wherein the anti-EGFRvIII agent is present in the solution at a concentration of about 75 ng/mL to about 500 ng/mL .

23. The method of claim 1, wherein the final volume of the aqueous solution is about 100 mL to about 300 mL solution.

24. The method of claim 1, wherein:
said EGFRvIII-binding domain comprises: (a) a heavy chain variable region (VH) that comprises: (i) a VH complementarity determining region one (CDR-H1) comprising the amino acid sequence of SEQ ID NO:3; (ii) a CDR-H2 comprising the amino acid sequence of SEQ ID NO:4; and (iii) a CDR-H3 comprising the amino acid sequence of SEQ ID NO:5; and (b) a light chain variable region (VL) that comprises: (i) a VL complementarity determining region one (CDR-L1) comprising the amino acid sequence of SEQ ID NO:6; (ii) a CDR-L2 comprising the amino acid sequence of SEQ ID NO:7;
and (iii) a CDR-L3 comprising the amino acid sequence of SEQ ID NO:8; and
said CD3-binding domain comprises: (a) a heavy chain variable region (VH) that comprises: (i) a VH complementarity determining region one (CDR-H1) comprising the amino acid sequence of SEQ ID NO:99; (ii) a CDR-H2 comprising the amino acid sequence of SEQ ID NO: 100; and (iii) a CDR-H3 comprising the amino acid sequence of SEQ ID NO: 101; and (b) a light chain variable region (VL) that comprises: (i) a VL complementarity determining region one (CDR-L1) comprising the amino acid sequence of SEQ ID NO:96; (ii) a CDR-L2 comprising the amino acid sequence of SEQ ID NO:97; and (iii) a CDR-L3 comprising the amino acid sequence of SEQ ID NO:98.

25. The method of claim 1, wherein said EGFRvIII-binding domain comprises: a VH that comprises the amino acid sequence of SEQ ID NO:9, and a VL that comprises the amino acid sequence of SEQ ID NO:10; and wherein said CD3-binding domain comprises: a VH that comprises the amino acid sequence of SEQ ID NO: 102, and a VL that comprises the amino acid sequence of SEQ ID NO: 103.

26. The method of claim 1, wherein said EGFRvIII-binding domain comprises the amino acid sequence of SEQ ID NO: 11, and said CD3-binding domain comprises the amino acid sequence of SEQ ID NO: 104.

27. The method of claim 1, wherein said anti-EGFRvIII agent comprises the amino acid sequence of SEQ ID NO: 12.

28. The method of claim 1, wherein said anti-EGFRvIII agent comprises the amino acid sequence of SEQ ID NO: 13.

29. A kit comprising an administration system and an anti-EGFRvIII agent, wherein one or more of the components of the administration system, or parts thereof, which contact the aqueous solution substantially lack a polyvinyl chloride (PVC) or PVC plasticizer and/or substantially lack air.

30. The kit of claim 29, wherein the administration system is an infusion system comprising an infusion line, wherein at least part of the infusion line substantially lacks PVC or a PVC plasticizer.

31. A kit comprising an IV bag comprising an aqueous solution comprising IVSS and saline and a container comprising an anti-EGFRvIII agent, wherein at least the parts of the IV bag which contact the aqueous solution substantially lack a polyvinyl chloride (PVC) or PVC plasticizer.


SEQUENCE LISTING (142 sequences)

SEQ ID NO: 1 - protein, 919 aa, Artificial Sequence
  Human EFGRvIII
LEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 2 - protein, 919 aa, Artificial Sequence
  Cynomolgus EGFRvIII
LEEKKGNYVVTDHGSCVRACGADSYEMEEDGVRKCKKCEGPCRKVCNGIGIGEFKDTLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSSQKTKIISNRGENSCKATGQVCHALCSPEGCWGPEPRDCVSCQNVSRGRECVDKCNILEGEPREFVENSECIQCHPECLPQVMNITCTGRGPDNCIQCAHYIDGPHCVKTCPAGVMGENNTLVWKYADAGHVCHLCHPNCTYGCTGPGLEGCARNGPKIPSIATGMLGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 3 - protein, 5 aa, Artificial Sequence
  EGFRvIII-binding domain CDR-H1
NYGMH

SEQ ID NO: 4 - protein, 17 aa, Artificial Sequence
  EGFRvIII- binding domain CDR-H2
VIWYDGSDKYYADSVRG

SEQ ID NO: 5 - protein, 15 aa, Artificial Sequence
  EGFRvIII- binding domain CDR-H3
DGYDILTGNPRDFDY

SEQ ID NO: 6 - protein, 16 aa, Artificial Sequence
  EGFRvIII- binding domain CDR-L1
RSSQSLVHSDGNTYLS

SEQ ID NO: 7 - protein, 7 aa, Artificial Sequence
  EGFRvIII- binding domain CDR-L2
RISRRFS

SEQ ID NO: 8 - protein, 9 aa, Artificial Sequence
  EGFRvIII- binding domain CDR-L3
MQSTHVPRT

SEQ ID NO: 9 - protein, 124 aa, Artificial Sequence
  EGFRvIII-binding domain VH
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSS

SEQ ID NO: 10 - protein, 112 aa, Artificial Sequence
  EGFRvIII- binding domain VL
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 11 - protein, 251 aa, Artificial Sequence
  EGFRvIII-binding domain scFv
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 12 - protein, 506 aa, Artificial Sequence
  EGFRvIII - CD3 bispecific molecule
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 13 - protein, 512 aa, Artificial Sequence
  EGFRvIII - CD3 bispecific molecule with his tag
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 14 - protein, 6 aa, Artificial Sequence
  Hexa-histidine
HHHHHH

SEQ ID NO: 15 - protein, 14 aa, Artificial Sequence
  CD3-binding domain CDR-L1 of F6A
GSSTGAVTSGYYPN

SEQ ID NO: 16 - protein, 7 aa, Artificial Sequence
  CD3-binding domain CDR-L2 of F6A
GTKFLAP

SEQ ID NO: 17 - protein, 9 aa, Artificial Sequence
  CD3-binding domain CDR-L3 of F6A
ALWYSNRWV

SEQ ID NO: 18 - protein, 5 aa, Artificial Sequence
  CD3-binding domain CDR-H1 of F6A
IYAMN

SEQ ID NO: 19 - protein, 19 aa, Artificial Sequence
  CD3-binding domain CDR-H2 of F6A
RIRSKYNNYATYYADSVKS

SEQ ID NO: 20 - protein, 14 aa, Artificial Sequence
  CD3-binding domain CDR-H3 of F6A
HGNFGNSYVSFFAY

SEQ ID NO: 21 - protein, 125 aa, Artificial Sequence
  CD3-binding domain VH of F6A
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 22 - protein, 109 aa, Artificial Sequence
  CD3-binding domain VL of F6A
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 23 - protein, 249 aa, Artificial Sequence
  CD3-binding domain scFv of F6A
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 24 - protein, 14 aa, Artificial Sequence
  CD3-binding domain CDR-L1 of H2C
GSSTGAVTSGYYPN

SEQ ID NO: 25 - protein, 7 aa, Artificial Sequence
  CD3-binding domain CDR-L2 of H2C
GTKFLAP

SEQ ID NO: 26 - protein, 9 aa, Artificial Sequence
  CD3-binding domain CDR-L3 of H2C
ALWYSNRWV

SEQ ID NO: 27 - protein, 5 aa, Artificial Sequence
  CD3-binding domain CDR-H1 of H2C
KYAMN

SEQ ID NO: 28 - protein, 19 aa, Artificial Sequence
  CD3-binding domain CDR-H2 of H2C
RIRSKYNNYATYYADSVKD

SEQ ID NO: 29 - protein, 14 aa, Artificial Sequence
  CD3-binding domain CDR-H3 of H2C
HGNFGNSYISYWAY

SEQ ID NO: 30 - protein, 125 aa, Artificial Sequence
  CD3-binding domain VH of H2C
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 31 - protein, 109 aa, Artificial Sequence; identical to SEQ ID NO: 22
  CD3-binding domain VL of H2C

SEQ ID NO: 32 - protein, 249 aa, Artificial Sequence
  CD3-binding domain scFv of H2C
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 33 - protein, 14 aa, Artificial Sequence
  CD3-binding domain CDR-L1 of H1E
GSSTGAVTSGYYPN

SEQ ID NO: 34 - protein, 7 aa, Artificial Sequence
  CD3-binding domain CDR-L2 of H1E
GTKFLAP

SEQ ID NO: 35 - protein, 9 aa, Artificial Sequence
  CD3-binding domain CDR-L3 of H1E
ALWYSNRWV

SEQ ID NO: 36 - protein, 5 aa, Artificial Sequence
  CD3-binding domain CDR-H1 of H1E
SYAMN

SEQ ID NO: 37 - protein, 19 aa, Artificial Sequence
  CD3-binding domain CDR-H2 of H1E
RIRSKYNNYATYYADSVKG

SEQ ID NO: 38 - protein, 14 aa, Artificial Sequence
  CD3-binding domain CDR-H3 of H1E
HGNFGNSYLSFWAY

SEQ ID NO: 39 - protein, 125 aa, Artificial Sequence
  CD3-binding domain VH of H1E
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSS

SEQ ID NO: 40 - protein, 109 aa, Artificial Sequence; identical to SEQ ID NO: 22
  CD3-binding domain VL of H1E

SEQ ID NO: 41 - protein, 249 aa, Artificial Sequence
  CD3-binding domain scFv of H1E
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 42 - protein, 14 aa, Artificial Sequence
  CD3-binding domain CDR-L1 of G4H
GSSTGAVTSGYYPN

SEQ ID NO: 43 - protein, 7 aa, Artificial Sequence
  CD3-binding domain CDR-L2 of G4H
GTKFLAP

SEQ ID NO: 44 - protein, 9 aa, Artificial Sequence
  CD3-binding domain CDR-L3 of G4H
ALWYSNRWV

SEQ ID NO: 45 - protein, 5 aa, Artificial Sequence
  CD3-binding domain CDR-H1 of G4H
RYAMN

SEQ ID NO: 46 - protein, 19 aa, Artificial Sequence
  CD3-binding domain CDR-H2 of G4H
RIRSKYNNYATYYADSVKG

SEQ ID NO: 47 - protein, 14 aa, Artificial Sequence
  CD3-binding domain CDR-H3 of G4H
HGNFGNSYLSYFAY

SEQ ID NO: 48 - protein, 125 aa, Artificial Sequence
  CD3-binding domain VH of G4H
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSS

SEQ ID NO: 49 - protein, 109 aa, Artificial Sequence; identical to SEQ ID NO: 22
  CD3-binding domain VL of G4H

SEQ ID NO: 50 - protein, 249 aa, Artificial Sequence
  CD3-binding domain scFv of G4H
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 51 - protein, 14 aa, Artificial Sequence
  CD3-binding domain CDR-L1 of A2J
RSSTGAVTSGYYPN

SEQ ID NO: 52 - protein, 7 aa, Artificial Sequence
  CD3-binding domain CDR-L2 of A2J
ATDMRPS

SEQ ID NO: 53 - protein, 9 aa, Artificial Sequence
  CD3-binding domain CDR-L3 of A2J
ALWYSNRWV

SEQ ID NO: 54 - protein, 5 aa, Artificial Sequence
  CD3-binding domain CDR-H1 of A2J
VYAMN

SEQ ID NO: 55 - protein, 19 aa, Artificial Sequence
  CD3-binding domain CDR-H2 of A2J
RIRSKYNNYATYYADSVKK

SEQ ID NO: 56 - protein, 14 aa, Artificial Sequence
  CD3-binding domain CDR-H3 of A2J
HGNFGNSYLSWWAY

SEQ ID NO: 57 - protein, 125 aa, Artificial Sequence
  CD3-binding domain VH of A2J
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSS

SEQ ID NO: 58 - protein, 109 aa, Artificial Sequence
  CD3-binding domain VL of A2J
QTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 59 - protein, 249 aa, Artificial Sequence
  CD3-binding domain scFv of A2J
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 60 - protein, 14 aa, Artificial Sequence
  CD3-binding domain CDR-L1 of E1L
GSSTGAVTSGYYPN

SEQ ID NO: 61 - protein, 7 aa, Artificial Sequence
  CD3-binding domain CDR-L2 of E1L
GTKFLAP

SEQ ID NO: 62 - protein, 9 aa, Artificial Sequence
  CD3-binding domain CDR-L3 of E1L
ALWYSNRWV

SEQ ID NO: 63 - protein, 5 aa, Artificial Sequence
  CD3-binding domain CDR-H1 of E1L
KYAMN

SEQ ID NO: 64 - protein, 19 aa, Artificial Sequence
  CD3-binding domain CDR-H2 of E1L
RIRSKYNNYATYYADSVKS

SEQ ID NO: 65 - protein, 14 aa, Artificial Sequence
  CD3-binding domain CDR-H3 of E1L
HGNFGNSYTSYYAY

SEQ ID NO: 66 - protein, 125 aa, Artificial Sequence
  CD3-binding domain VH of E1L
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSS

SEQ ID NO: 67 - protein, 109 aa, Artificial Sequence; identical to SEQ ID NO: 22
  CD3-binding domain VL of E1L

SEQ ID NO: 68 - protein, 249 aa, Artificial Sequence
  CD3-binding domain scFv of E1L
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 69 - protein, 14 aa, Artificial Sequence
  CD3-binding domain CDR-L1 of E2M
RSSTGAVTSGYYPN

SEQ ID NO: 70 - protein, 7 aa, Artificial Sequence
  CD3-binding domain CDR-L2 of E2M
ATDMRPS

SEQ ID NO: 71 - protein, 9 aa, Artificial Sequence
  CD3-binding domain CDR-L3 of E2M
ALWYSNRWV

SEQ ID NO: 72 - protein, 5 aa, Artificial Sequence
  CD3-binding domain CDR-H1 of E2M
GYAMN

SEQ ID NO: 73 - protein, 19 aa, Artificial Sequence
  CD3-binding domain CDR-H2 of E2M
RIRSKYNNYATYYADSVKE

SEQ ID NO: 74 - protein, 14 aa, Artificial Sequence
  CD3-binding domain CDR-H3 of E2M
HRNFGNSYLSWFAY

SEQ ID NO: 75 - protein, 125 aa, Artificial Sequence
  CD3-binding domain VH of E2M
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSS

SEQ ID NO: 76 - protein, 109 aa, Artificial Sequence; identical to SEQ ID NO: 58
  CD3-binding domain VL of E2M

SEQ ID NO: 77 - protein, 249 aa, Artificial Sequence
  CD3-binding domain scFv of E2M
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 78 - protein, 14 aa, Artificial Sequence
  CD3-binding domain CDR-L1 of F7O
GSSTGAVTSGYYPN

SEQ ID NO: 79 - protein, 7 aa, Artificial Sequence
  CD3-binding domain CDR-L2 of F7O
GTKFLAP

SEQ ID NO: 80 - protein, 9 aa, Artificial Sequence
  CD3-binding domain CDR-L3 of F7O
ALWYSNRWV

SEQ ID NO: 81 - protein, 5 aa, Artificial Sequence
  CD3-binding domain CDR-H1 of F7O
VYAMN

SEQ ID NO: 82 - protein, 19 aa, Artificial Sequence
  CD3-binding domain CDR-H2 of F7O
RIRSKYNNYATYYADSVKK

SEQ ID NO: 83 - protein, 14 aa, Artificial Sequence
  CD3-binding domain CDR-H3 of F7O
HGNFGNSYISWWAY

SEQ ID NO: 84 - protein, 125 aa, Artificial Sequence
  CD3-binding domain VH of F7O
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSS

SEQ ID NO: 85 - protein, 109 aa, Artificial Sequence; identical to SEQ ID NO: 22
  CD3-binding domain VL of F7O

SEQ ID NO: 86 - protein, 249 aa, Artificial Sequence
  CD3-binding domain scFv of F7O
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 87 - protein, 14 aa, Artificial Sequence
  CD3-binding domain CDR-L1 of F12Q
GSSTGAVTSGNYPN

SEQ ID NO: 88 - protein, 7 aa, Artificial Sequence
  CD3-binding domain CDR-L2 of F12Q
GTKFLAP

SEQ ID NO: 89 - protein, 9 aa, Artificial Sequence
  CD3-binding domain CDR-L3 of F12Q
VLWYSNRWV

SEQ ID NO: 90 - protein, 5 aa, Artificial Sequence
  CD3-binding domain CDR-H1 of F12Q
SYAMN

SEQ ID NO: 91 - protein, 19 aa, Artificial Sequence
  CD3-binding domain CDR-H2 of F12Q
RIRSKYNNYATYYADSVKG

SEQ ID NO: 92 - protein, 14 aa, Artificial Sequence
  CD3-binding domain CDR-H3 of F12Q
HGNFGNSYVSWWAY

SEQ ID NO: 93 - protein, 125 aa, Artificial Sequence
  CD3-binding domain VH of F12Q
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSS

SEQ ID NO: 94 - protein, 109 aa, Artificial Sequence
  CD3-binding domain VL of F12Q
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 95 - protein, 249 aa, Artificial Sequence
  CD3-binding domain scFv of F12Q
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 96 - protein, 14 aa, Artificial Sequence
  CD3-binding domain CDR-L1 of I2C
GSSTGAVTSGNYPN

SEQ ID NO: 97 - protein, 7 aa, Artificial Sequence
  CD3-binding domain CDR-L2 of I2C
GTKFLAP

SEQ ID NO: 98 - protein, 9 aa, Artificial Sequence
  CD3-binding domain CDR-L3 of I2C
VLWYSNRWV

SEQ ID NO: 99 - protein, 5 aa, Artificial Sequence
  CD3-binding domain CDR-H1 of I2C
KYAMN

SEQ ID NO: 100 - protein, 19 aa, Artificial Sequence
  CD3-binding domain CDR-H2 of I2C
RIRSKYNNYATYYADSVKD

SEQ ID NO: 101 - protein, 14 aa, Artificial Sequence
  CD3-binding domain CDR-H3 of I2C
HGNFGNSYISYWAY

SEQ ID NO: 102 - protein, 125 aa, Artificial Sequence; identical to SEQ ID NO: 30
  CD3-binding domain VH of I2C

SEQ ID NO: 103 - protein, 109 aa, Artificial Sequence; identical to SEQ ID NO: 94
  CD3-binding domain VL of I2C

SEQ ID NO: 104 - protein, 249 aa, Artificial Sequence
  CD3-binding domain scFv of I2C
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 105 - protein, 14 aa, Artificial Sequence
  CD3-binding domain CDR-L1 of F12q
GSSTGAVTSGNYPN

SEQ ID NO: 106 - protein, 7 aa, Artificial Sequence
  CD3-binding domain CDR-L2 of F12q
GTKFLAP

SEQ ID NO: 107 - protein, 9 aa, Artificial Sequence
  CD3-binding domain CDR-L3 of F12q
VLWYSNRWV

SEQ ID NO: 108 - protein, 5 aa, Artificial Sequence
  CD3-binding domain CDR-H1 of F12q
SYAMN

SEQ ID NO: 109 - protein, 19 aa, Artificial Sequence
  CD3-binding domain CDR-H2 of F12q
RIRSKYNNYATYYADSVKG

SEQ ID NO: 110 - protein, 14 aa, Artificial Sequence
  CD3-binding domain CDR-H3 of F12q
HGNFGNSYVSWWAY

SEQ ID NO: 111 - protein, 125 aa, Artificial Sequence
  CD3-binding domain VH of F12q
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSS

SEQ ID NO: 112 - protein, 109 aa, Artificial Sequence; identical to SEQ ID NO: 94
  CD3-binding domain VL of F12q

SEQ ID NO: 113 - protein, 249 aa, Artificial Sequence
  CD3-binding domain scFv of F12q
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 114 - protein, 4 aa, Artificial Sequence
  Peptide linker
GGGG

SEQ ID NO: 115 - protein, 5 aa, Artificial Sequence
  Peptide linker
GGGGS

SEQ ID NO: 116 - protein, 5 aa, Artificial Sequence
  Peptide linker
GGGGQ

SEQ ID NO: 117 - protein, 6 aa, Artificial Sequence
  Peptide linker
PGGGGS

SEQ ID NO: 118 - protein, 6 aa, Artificial Sequence
  Peptide linker
PGGDGS

SEQ ID NO: 119 - protein, 6 aa, Artificial Sequence
  Peptide linker
SGGGGS

SEQ ID NO: 120 - protein, 9 aa, Artificial Sequence
  Peptide linker
GGGGSGGGS

SEQ ID NO: 121 - protein, 10 aa, Artificial Sequence
  Peptide linker
GGGGSGGGGS

SEQ ID NO: 122 - protein, 15 aa, Artificial Sequence
  Peptide linker
GGGGSGGGGSGGGGS

SEQ ID NO: 123 - protein, 105 aa, Artificial Sequence
  Human CD3 epsilon extracellular domain
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 124 - protein, 96 aa, Artificial Sequence
  Callithrix jacchus CD3 epsilon extracellular domain
QDGNEEMGDTTQNPYKVSISGTTVTLTCPRYDGHEIKWLVNSQNKEGHEDHLLLEDFSEMEQSGYYACLSKETPAEEASHYLYLKARVCENCVEVD

SEQ ID NO: 125 - protein, 96 aa, Artificial Sequence; identical to SEQ ID NO: 124
  Saguinus oedipus CD3 epsilon extracellular domain

SEQ ID NO: 126 - protein, 96 aa, Artificial Sequence
  Saimiri sciureus CD3 epsilon extracellular domain
QDGNEEIGDTTQNPYKVSISGTTVTLTCPRYDGQEIKWLVNDQNKEGHEDHLLLEDFSEMEQSGYYACLSKETPTEEASHYLYLKARVCENCVEVD

SEQ ID NO: 127 - protein, 5 aa, Artificial Sequence
  EGFRvIII antibody 1 CDR-H1
SYGMH

SEQ ID NO: 128 - protein, 17 aa, Artificial Sequence
  EGFRvIII antibody 1 CDR-H2
VIWYDGSNKYYVDSVKG

SEQ ID NO: 129 - protein, 11 aa, Artificial Sequence
  EGFRvIII antibody 1 CDR-H3
DGWQQLAPFDY

SEQ ID NO: 130 - protein, 16 aa, Artificial Sequence
  EGFRvIII antibody 1 CDR-L1
RSSQSLVHSDGNTYLS

SEQ ID NO: 131 - protein, 7 aa, Artificial Sequence
  EGFRvIII antibody 1 CDR-L2
KISNRFS

SEQ ID NO: 132 - protein, 9 aa, Artificial Sequence
  EGFRvIII antibody 1 CDR-L3
MQATQLPRT

SEQ ID NO: 133 - protein, 120 aa, Artificial Sequence
  EGFRvIII antibody 1 VH
QVQLVESGGGVVQPGRSLRLSCAASGFTFSSYGMHWVRQAPGKGLEWVAVIWYDGSNKYYVDSVKGRFTISRDNSKNTLYLQMNSLRAEDTAVYYCARDGWQQLAPFDYWGQGTLVTVSA

SEQ ID NO: 134 - protein, 112 aa, Artificial Sequence
  EGFRvIII antibody 1 VL
DIVMTQTPLSSPVTLGQPASISCRSSQSLVHSDGNTYLSWLHQRPGQPPRLLIYKISNRFSGVPDRFSGSGAGTAFTLKISRVEAEDVGVYYCMQATQLPRTFGQGTKVEIK

SEQ ID NO: 135 - protein, 324 aa, Artificial Sequence
  EGFRvIII antibody 1 CH
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 136 - protein, 107 aa, Artificial Sequence
  EGFRvIII antibody 1 CL
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 137 - protein, 444 aa, Artificial Sequence
  EGFRvIII antibody 1 heavy chain
QVQLVESGGGVVQPGRSLRLSCAASGFTFSSYGMHWVRQAPGKGLEWVAVIWYDGSNKYYVDSVKGRFTISRDNSKNTLYLQMNSLRAEDTAVYYCARDGWQQLAPFDYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 138 - protein, 219 aa, Artificial Sequence
  EGFRvIII antibody 1 light chain
DIVMTQTPLSSPVTLGQPASISCRSSQSLVHSDGNTYLSWLHQRPGQPPRLLIYKISNRFSGVPDRFSGSGAGTAFTLKISRVEAEDVGVYYCMQATQLPRTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 139 - protein, 124 aa, Artificial Sequence
  EGFRvIII antibody 2 VH
QVQLVESGGGVVQSGRSLRLSCAASGFTFRNYGMHWVRQAPGKGLEWVAVIWYDGSDKYYADSVRGRFTISRDNSKNTLYLQMNSLRAEDTAVYYCARDGYDILTGNPRDFDYWGQGTLVTVSA

SEQ ID NO: 140 - protein, 112 aa, Artificial Sequence
  EGFRvIII antibody 2 VL
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGQGTKVEIK

SEQ ID NO: 141 - protein, 448 aa, Artificial Sequence
  EGFRvIII antibody 2 heavy chain
QVQLVESGGGVVQSGRSLRLSCAASGFTFRNYGMHWVRQAPGKGLEWVAVIWYDGSDKYYADSVRGRFTISRDNSKNTLYLQMNSLRAEDTAVYYCARDGYDILTGNPRDFDYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 142 - protein, 219 aa, Artificial Sequence
  EGFRvIII antibody 2 light chain
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC